SEQUENCE LISTING (132 sequences)

SEQ ID NO: 1 - protein, 8 aa, Homo sapiens
GIDFSNYG

SEQ ID NO: 2 - protein, 8 aa, Homo sapiens
IYPDYGAT

SEQ ID NO: 3 - protein, 11 aa, Homo sapiens
ARSTGWGDFNL

SEQ ID NO: 4 - protein, 8 aa, Homo sapiens
GIDFSSAG

SEQ ID NO: 5 - protein, 8 aa, Homo sapiens
IYPDYGVT

SEQ ID NO: 6 - protein, 14 aa, Homo sapiens
ARMDAADAGYGYDL

SEQ ID NO: 7 - protein, 9 aa, Homo sapiens
GFSFSSYYF

SEQ ID NO: 8 - protein, 9 aa, Homo sapiens
IYAGTSANT

SEQ ID NO: 9 - protein, 23 aa, Homo sapiens
ARDRGYYTYADAGYAYATDGFDL

SEQ ID NO: 10 - protein, 9 aa, Homo sapiens
GFSFSDTAY

SEQ ID NO: 11 - protein, 7 aa, Homo sapiens
IAFGDGR

SEQ ID NO: 12 - protein, 6 aa, Homo sapiens
ASNFTL

SEQ ID NO: 13 - protein, 8 aa, Homo sapiens
GFSFSSTA

SEQ ID NO: 14 - protein, 7 aa, Homo sapiens
IGFSSGI

SEQ ID NO: 15 - protein, 6 aa, Homo sapiens
ARFFNL

SEQ ID NO: 16 - protein, 9 aa, Homo sapiens
GFSFSYSYY

SEQ ID NO: 17 - protein, 9 aa, Homo sapiens
IYGGGSGSS

SEQ ID NO: 18 - protein, 11 aa, Homo sapiens
ARIDDAYATVM

SEQ ID NO: 19 - protein, 8 aa, Homo sapiens
GYTFTSYY

SEQ ID NO: 20 - protein, 8 aa, Homo sapiens
INPSGGST

SEQ ID NO: 21 - protein, 24 aa, Homo sapiens
ARDRPGRSSIAAAGPKRGHYGMDV

SEQ ID NO: 22 - protein, 8 aa, Homo sapiens
GVSVSNNY

SEQ ID NO: 23 - protein, 7 aa, Homo sapiens
MYSDATT

SEQ ID NO: 24 - protein, 11 aa, Homo sapiens
ARESVAGTLDF

SEQ ID NO: 25 - protein, 8 aa, Homo sapiens
GYTFTSYY

SEQ ID NO: 26 - protein, 8 aa, Homo sapiens
INPSGGST

SEQ ID NO: 27 - protein, 12 aa, Homo sapiens
ARSLSSGSYYDY

SEQ ID NO: 28 - protein, 6 aa, Homo sapiens
QNIGSN

SEQ ID NO: 29 - protein, 11 aa, Homo sapiens
QCTYYGSSYVA

SEQ ID NO: 30 - protein, 6 aa, Homo sapiens
QSVGSN

SEQ ID NO: 31 - protein, 12 aa, Homo sapiens
HSYYYSSTSADA

SEQ ID NO: 32 - protein, 6 aa, Homo sapiens
QSVGTN

SEQ ID NO: 33 - protein, 12 aa, Homo sapiens
HSYYYSSTSADA

SEQ ID NO: 34 - protein, 6 aa, Homo sapiens
QNIYSN

SEQ ID NO: 35 - protein, 15 aa, Homo sapiens
QSNYYSSSSNYGPNA

SEQ ID NO: 36 - protein, 6 aa, Homo sapiens
QSIGNN

SEQ ID NO: 37 - protein, 9 aa, Homo sapiens
QCTYASSAT

SEQ ID NO: 38 - protein, 8 aa, Homo sapiens
ETVYSNDR

SEQ ID NO: 39 - protein, 10 aa, Homo sapiens
AGGYNGNIWA

SEQ ID NO: 40 - protein, 6 aa, Homo sapiens
EDIYSS

SEQ ID NO: 41 - protein, 13 aa, Homo sapiens
QNYYYVRGADYWA

SEQ ID NO: 42 - protein, 9 aa, Homo sapiens
SSDVGGYNY

SEQ ID NO: 43 - protein, 10 aa, Homo sapiens
SSYTSSRTLV

SEQ ID NO: 44 - protein, 8 aa, Homo sapiens
SSNIGTYP

SEQ ID NO: 45 - protein, 11 aa, Homo sapiens
ATWGDSLNAVV

SEQ ID NO: 46 - protein, 9 aa, Homo sapiens
SSDVGGYNY

SEQ ID NO: 47 - protein, 10 aa, Homo sapiens
SSYTSSRTLV

SEQ ID NO: 48 - protein, 131 aa, Homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARDRPGRSSIAAAGPKRGHYGMDVWGQGTTVSVSS

SEQ ID NO: 49 - protein, 117 aa, Homo sapiens
QVQLVESGGGLIQPGGSLRLSCAVSGVSVSNNYMNWVRQAPGKGLEWVSSMYSDATTHYADSVKGRFTISRDNSKNNMYLQMDSLRAEDTAVYYCARESVAGTLDFWGQGTTVSVSS

SEQ ID NO: 50 - protein, 119 aa, Homo sapiens
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARSLSSGSYYDYWGQGTLVSVSS

SEQ ID NO: 51 - protein, 118 aa, Homo sapiens
QEQLVESGGGLVTPGGTLTLTCTASGIDFSNYGISWVRQAPGKGLEWIGYIYPDYGATAYANWVNGRFTVSLDNAQNTVTLNMTSLTAADTATYFCARSTGWGDFNLWGPGTLVTVSS

SEQ ID NO: 52 - protein, 121 aa, Homo sapiens
QKQLVESGGGLVKPEGSLTLTCKVSGIDFSSAGISWVRQAPGKGLEWIAYIYPDYGVTDYANWVNGRFTISLDNAQNTVFLQMTSLTAADTATYFCARMDAADAGYGYDLWGPGTLVTVSS

SEQ ID NO: 53 - protein, 130 aa, Homo sapiens
QSVEESGGDLVKSGASLTLTCTASGFSFSSYYFMCWVRQAPGRGLEWIACIYAGTSANTYYASWAKGRFTISKTSSTTVTLQMTSLTAADTATYFCARDRGYYTYADAGYAYATDGFDLWGPGTLVTVSS

SEQ ID NO: 54 - protein, 113 aa, Homo sapiens
QEQLVESGGGLVKPGASLTLTCTASGFSFSDTAYICWVRQAPGKGLEWVACIAFGDGRTYLASWAKGRFTISKTSSTTVTLQMTSLPAADTATYFCASNFTLWGPGTLVTVSS

SEQ ID NO: 55 - protein, 111 aa, Homo sapiens
QSVEESGGDLVKPGASLTLTCTASGFSFSSTAMCWVRQAPGKGLEWIACIGFSSGIINYANSVKGRFTISKTSSTTVSLQMNSLTAADTATYFCARFFNLWGPGTLVTVSS

SEQ ID NO: 56 - protein, 119 aa, Homo sapiens
QKQLVESGGDLVKPEGSLTLTCTASGFSFSYSYYMCWVRQAPGKGLEWIGCIYGGGSGSSYYASWAKGRFTISKTSSTTVTLQVTSLTAADTATYFCARIDDAYATVMWGPGTLVTVSS

SEQ ID NO: 57 - protein, 109 aa, Homo sapiens
DVVMTQTPASVSEPVGGTVTIKCQASQNIGSNLAWYQQKPGQPPNLLIYGASNLASGVSSRFKGSSSGTEYTLTISDLECADAATYYCQCTYYGSSYVAFGGGTEVVVK

SEQ ID NO: 58 - protein, 110 aa, Homo sapiens
AAVLTQTTSPVSGAVGGTVTIKCQASQSVGSNLAWYQQKPGQPPKILIGGASKLASGVPSRFSGSGSGTEFTLTISDLECADAGTYYCHSYYYSSTSADAFGGGTKVVVE

SEQ ID NO: 59 - protein, 110 aa, Homo sapiens
DVVMTQTTSPVSGAVGGTVTIKCQASQSVGTNLAWYQQKPGQPPKILIGGASKLASGVPSRFSGSGSGTEFTFTISDLECADAGTYYCHSYYYSSTSADAFGGGTEVVVK

SEQ ID NO: 60 - protein, 113 aa, Homo sapiens
ALVMTQTPASVEAAVGGTVTIKCQASQNIYSNLAWYQQKPGQPPKRLIYKASTLPSGVPSRFKGSGSGTQFTLTISDLECADAATYYCQSNYYSSSSNYGPNAFGGGTEVVVK

SEQ ID NO: 61 - protein, 107 aa, Homo sapiens
DVVMTQTPASVSEPVGGTVTINCQAIQSIGNNFVWYQQKPGQPPKLLIYKASIRASGVSSRFRGSGSGTDFTLTISDLECDDTGTYYCQCTYASSATFGGGTEVVVK

SEQ ID NO: 62 - protein, 110 aa, Homo sapiens
DPVLTQTPSSVSAAVGGTVTISCQATETVYSNDRLAWYQQKPGQPPKLLIYRISTLESGVPSRFKGSGSGTQFTLTISGVQCDDAATYYCAGGYNGNIWAFGGGTEVVVK

SEQ ID NO: 63 - protein, 111 aa, Homo sapiens
DVVMTQTASPVSGAVGGTVTIKCQASEDIYSSLAWYQQKPGQPPKLLIYQASKLASGVPPRFSGSGSGTEYTLTISDLECADAATYYCQNYYYVRGADYWAFGGGTEVVVE

SEQ ID NO: 64 - protein, 110 aa, Homo sapiens
QSALNQPASVSGSPGQSITISCTGTSSDVGGYNYVSWYQQHPGKAPKLMIYEVSNRPSGVSNRFSGSKSGNTASLTISGLQAEDEADYYCSSYTSSRTLVFGTGTKVTVL

SEQ ID NO: 65 - protein, 110 aa, Homo sapiens
SYELTQPPSASGTPGQRVTISCSGSSSNIGTYPVNWYQKLPGTAPKLLIYTNNQRPSGVPDRFSGSKSGTSASLTISGLQSEDEADYYCATWGDSLNAVVFGGGTKVTVL

SEQ ID NO: 66 - protein, 110 aa, Homo sapiens
QSALTQPASVSGSPGQSITISCTGTSSDVGGYNYVSWYQQHPGKAPKLMIYDVTKRPSGVSNRFSGSKSGNTASLTVSGLQAEDEADYYCSSYTSSRTLVFGGGTKVTVL

SEQ ID NO: 67 - DNA, 24 bp, Homo sapiens
  source 1..24: mol_type = other DNA
ggaatcgacttcagtaactatggc

SEQ ID NO: 68 - DNA, 24 bp, Homo sapiens
  source 1..24: mol_type = other DNA
atttatcctgattatggtgccaca

SEQ ID NO: 69 - DNA, 33 bp, Homo sapiens
  source 1..33: mol_type = other DNA
gcaagaagtactggctggggtgactttaacttg

SEQ ID NO: 70 - DNA, 24 bp, Homo sapiens
  source 1..24: mol_type = other DNA
ggaatcgacttcagtagcgctggc

SEQ ID NO: 71 - DNA, 24 bp, Homo sapiens
  source 1..24: mol_type = other DNA
atttatcctgattatggtgtcaca

SEQ ID NO: 72 - DNA, 42 bp, Homo sapiens
  source 1..42: mol_type = other DNA
gcaagaatggatgctgctgatgctggttatggttatgacttg

SEQ ID NO: 73 - DNA, 27 bp, Homo sapiens
  source 1..27: mol_type = other DNA
ggattctccttcagtagctactacttc

SEQ ID NO: 74 - DNA, 27 bp, Homo sapiens
  source 1..27: mol_type = other DNA
atttatgctggtactagtgctaacact

SEQ ID NO: 75 - DNA, 69 bp, Homo sapiens
  source 1..69: mol_type = other DNA
gcgagagatcggggttactatacttatgctgatgctggttatgcttatgcaactgatggttttgatctc

SEQ ID NO: 76 - DNA, 27 bp, Homo sapiens
  source 1..27: mol_type = other DNA
ggattctccttcagcgacaccgcgtac

SEQ ID NO: 77 - DNA, 21 bp, Homo sapiens
  source 1..21: mol_type = other DNA
attgcttttggtgatggcagg

SEQ ID NO: 78 - DNA, 18 bp, Homo sapiens
  source 1..18: mol_type = other DNA
gcgagtaattttacattg

SEQ ID NO: 79 - DNA, 24 bp, Homo sapiens
  source 1..24: mol_type = other DNA
ggattctccttcagtagcactgca

SEQ ID NO: 80 - DNA, 21 bp, Homo sapiens
  source 1..21: mol_type = other DNA
attggttttagtagtggtatt

SEQ ID NO: 81 - DNA, 18 bp, Homo sapiens
  source 1..18: mol_type = other DNA
gcgagattctttaatttg

SEQ ID NO: 82 - DNA, 27 bp, Homo sapiens
  source 1..27: mol_type = other DNA
ggattctctttcagttacagctactac

SEQ ID NO: 83 - DNA, 27 bp, Homo sapiens
  source 1..27: mol_type = other DNA
atttatggtggtggtagtggtagttct

SEQ ID NO: 84 - DNA, 33 bp, Homo sapiens
  source 1..33: mol_type = other DNA
gcgagaattgatgatgcttatgctactgtcatg

SEQ ID NO: 85 - DNA, 24 bp, Homo sapiens
  source 1..24: mol_type = other DNA
ggatacaccttcaccagctactat

SEQ ID NO: 86 - DNA, 24 bp, Homo sapiens
  source 1..24: mol_type = other DNA
atcaaccctagtggtggtagcaca

SEQ ID NO: 87 - DNA, 72 bp, Homo sapiens
  source 1..72: mol_type = other DNA
gcgagagatcgaccgggccgttccagtatagcagcagctgggccgaagcggggccactacggtatggacgtc

SEQ ID NO: 88 - DNA, 24 bp, Homo sapiens
  source 1..24: mol_type = other DNA
ggggtcagcgtcagtaacaactac

SEQ ID NO: 89 - DNA, 21 bp, Homo sapiens
  source 1..21: mol_type = other DNA
atgtatagtgatgccactaca

SEQ ID NO: 90 - DNA, 33 bp, Homo sapiens
  source 1..33: mol_type = other DNA
gcgagggaatcagtggctggcacgcttgacttc

SEQ ID NO: 91 - DNA, 24 bp, Homo sapiens
  source 1..24: mol_type = other DNA
ggatacaccttcaccagctactat

SEQ ID NO: 92 - DNA, 24 bp, Homo sapiens
  source 1..24: mol_type = other DNA
atcaaccctagtggtggtagcaca

SEQ ID NO: 93 - DNA, 36 bp, Homo sapiens
  source 1..36: mol_type = other DNA
gcgagatccctgagcagtgggagctactatgactac

SEQ ID NO: 94 - DNA, 18 bp, Homo sapiens
  source 1..18: mol_type = other DNA
caggtgcagctggtgcag

SEQ ID NO: 95 - DNA, 33 bp, Homo sapiens
  source 1..33: mol_type = other DNA
caatgtacttattatggtagtagttatgttgct

SEQ ID NO: 96 - DNA, 18 bp, Homo sapiens
  source 1..18: mol_type = other DNA
cagagcgttggtagtaat

SEQ ID NO: 97 - DNA, 36 bp, Homo sapiens
  source 1..36: mol_type = other DNA
cacagttattattatagtagtactagtgctgatgct

SEQ ID NO: 98 - DNA, 18 bp, Homo sapiens
  source 1..18: mol_type = other DNA
cagagcgttggtactaat

SEQ ID NO: 99 - DNA, 36 bp, Homo sapiens; identical to SEQ ID NO: 97
  source 1..36: mol_type = other DNA

SEQ ID NO: 100 - DNA, 18 bp, Homo sapiens
  source 1..18: mol_type = other DNA
cagaacatttacagcaat

SEQ ID NO: 101 - DNA, 45 bp, Homo sapiens
  source 1..45: mol_type = other DNA
caaagcaattattatagtagtagtagtaattatggccctaatgct

SEQ ID NO: 102 - DNA, 18 bp, Homo sapiens
  source 1..18: mol_type = other DNA
cagagcattggtaataat

SEQ ID NO: 103 - DNA, 27 bp, Homo sapiens
  source 1..27: mol_type = other DNA
caatgtacttatgctagtagtgctact

SEQ ID NO: 104 - DNA, 24 bp, Homo sapiens
  source 1..24: mol_type = other DNA
gagactgtttatagtaacgaccgc

SEQ ID NO: 105 - DNA, 30 bp, Homo sapiens
  source 1..30: mol_type = other DNA
gcaggcggttataatggtaatatttgggct

SEQ ID NO: 106 - DNA, 18 bp, Homo sapiens
  source 1..18: mol_type = other DNA
gaggacatttacagctct

SEQ ID NO: 107 - DNA, 39 bp, Homo sapiens
  source 1..39: mol_type = other DNA
cagaactattattatgttcgtggtgctgattattgggct

SEQ ID NO: 108 - DNA, 27 bp, Homo sapiens
  source 1..27: mol_type = other DNA
agcagtgacgttggtggttataactat

SEQ ID NO: 109 - DNA, 30 bp, Homo sapiens
  source 1..30: mol_type = other DNA
agctcatatacaagcagccgcactctcgtc

SEQ ID NO: 110 - DNA, 24 bp, Homo sapiens
  source 1..24: mol_type = other DNA
agctccaacatcggaacttatcct

SEQ ID NO: 111 - DNA, 33 bp, Homo sapiens
  source 1..33: mol_type = other DNA
gcaacatggggtgacagcctgaatgctgtggta

SEQ ID NO: 112 - DNA, 27 bp, Homo sapiens
  source 1..27: mol_type = other DNA
agcagtgacgttggtggttataactat

SEQ ID NO: 113 - DNA, 30 bp, Homo sapiens
  source 1..30: mol_type = other DNA
agctcatatacaagcagtagaactttagtc

SEQ ID NO: 114 - DNA, 393 bp, Homo sapiens
  source 1..393: mol_type = other DNA
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagagatcgaccgggccgttccagtatagcagcagctgggccgaagcggggccactacggtatggacgtctggggccaagggaccacggtcagcgtctcctca

SEQ ID NO: 115 - DNA, 351 bp, Homo sapiens
  source 1..351: mol_type = other DNA
caggtgcagctggtggagtctggaggaggcttgatccagcctggggggtccctaagactctcctgtgccgtctctggggtcagcgtcagtaacaactacatgaactgggtccgccaggctccagggaaggggctggagtgggtctcaagtatgtatagtgatgccactacacactatgcagactccgtgaagggccgattcaccatctccagagacaattccaagaacaatatgtatcttcaaatggacagcctgagagccgaagacacggccgtatattactgtgcgagggaatcagtggctggcacgcttgacttctggggccaggggaccacggtcagcgtctcctca

SEQ ID NO: 116 - DNA, 357 bp, Homo sapiens
  source 1..357: mol_type = other DNA
gaggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggtagagtcaccatgaccagggacacgtccacgagcacagtctacatggagctgagcagcctgagatctgaggacacggccgtgtattactgtgcgagatccctgagcagtgggagctactatgactactggggccagggaaccctggtcagcgtctcctca

SEQ ID NO: 117 - DNA, 354 bp, Homo sapiens
  source 1..354: mol_type = other DNA
caggagcagctggtggagtctggagggggtctggtcacgcctggaggaaccctgacactcacctgcacagcctctggaatcgacttcagtaactatggcattagctgggtccgccaggctccagggaaggggctggagtggatcggatacatttatcctgattatggtgccacagcctacgcgaactgggtgaatggccgattcaccgtctccctcgacaacgcccagaacacggtgactctgaatatgaccagtctgacagccgcggacacggccacctatttctgtgcaagaagtactggctggggtgactttaacttgtggggcccaggcaccctggtcaccgtctcttca

SEQ ID NO: 118 - DNA, 363 bp, Homo sapiens
  source 1..363: mol_type = other DNA
cagaagcagctggtggagtctggagggggtctggtcaagcctgagggatccctgacactcacctgcaaagtctctggaatcgacttcagtagcgctggcattagctgggtccgccaggctcccgggaaggggctggagtggatcgcatatatttatcctgattatggtgtcacagactacgcgaactgggtgaatggccgattcaccatctccctcgacaacgcccagaacacggtgtttctgcaaatgaccagtctgacagccgcggacactgccacctatttctgtgcaagaatggatgctgctgatgctggttatggttatgacttgtggggcccaggcaccctggtcactgtctcctca

SEQ ID NO: 119 - DNA, 390 bp, Homo sapiens
  source 1..390: mol_type = other DNA
cagtcggtggaggagtccgggggagacctggtcaagtctggggcatccctgacactcacctgcacagcctctggattctccttcagtagctactacttcatgtgctgggtccgccaggctccagggaggggtctggagtggatcgcatgcatttatgctggtactagtgctaacacttactacgcgagctgggcgaaaggccgattcaccatctccaaaacctcgtcgaccacggtgactctgcaaatgaccagtctgacagccgcggacacggccacctatttctgtgcgagagatcggggttactatacttatgctgatgctggttatgcttatgcaactgatggttttgatctctggggcccaggcaccctggtcaccgtctcttca

SEQ ID NO: 120 - DNA, 339 bp, Homo sapiens
  source 1..339: mol_type = other DNA
caggagcagctggtggagtccgggggaggcctggtcaagcctggggcatccctgacactcacctgcacagcttctggattctccttcagcgacaccgcgtacatatgttgggtccgccaggctccagggaaggggctggagtgggtcgcatgcattgcttttggtgatggcaggacatacttggcgagctgggcgaaaggccgattcaccatctccaaaacctcgtcgaccacggtgactctgcaaatgaccagtctgccagccgcggacacggccacctatttctgtgcgagtaattttacattgtggggcccaggcaccctggtcaccgtctcttca

SEQ ID NO: 121 - DNA, 333 bp, Homo sapiens
  source 1..333: mol_type = other DNA
cagtcggtggaggagtccgggggagacctggtcaagcctggggcatccctgacactcacctgcacagcctctggattctccttcagtagcactgcaatgtgctgggtccgccaggctccagggaaggggctggagtggatcgcttgcattggttttagtagtggtattattaactacgcgaactctgtgaagggccgattcaccatctccaagacctcgtcgaccacggtgtctctgcaaatgaacagtctgacagccgcggacacggccacctatttctgtgcgagattctttaatttgtggggcccaggcaccctggtcaccgtctcttca

SEQ ID NO: 122 - DNA, 357 bp, Homo sapiens
  source 1..357: mol_type = other DNA
cagaagcagctggtggagtccgggggagacctggtcaagcctgagggatccctgacactcacctgcacagcctctggattctctttcagttacagctactacatgtgctgggtccgccaggctccagggaaggggctggagtggatcggatgcatttatggtggtggtagtggtagttcttactacgcgagctgggcgaaaggccgattcaccatctccaaaacctcgtcgaccacggtgactctgcaagtgaccagtctgacagccgcggacacggccacctatttctgtgcgagaattgatgatgcttatgctactgtcatgtggggcccaggcaccctcgtcaccgtctcttca

SEQ ID NO: 123 - DNA, 330 bp, Homo sapiens
  source 1..330: mol_type = other DNA
cagtctgccctgaatcagcctgcctccgtgtctgggtctcctggacagtcgatcaccatctcctgcactggaaccagcagtgacgttggtggttataactatgtctcctggtaccaacagcacccaggcaaagcccccaaactcatgatttatgaggtcagtaatcggccctcaggggtttctaatcgcttctctggctccaagtctggcaacacggcctccctgaccatctctgggctccaggctgaggacgaggctgattattactgcagctcatatacaagcagccgcactctcgtcttcggaactgggaccaaggtcaccgtccta

SEQ ID NO: 124 - DNA, 330 bp, Homo sapiens
  source 1..330: mol_type = other DNA
tcctatgagctgactcagccaccctcagcgtctgggacccccgggcagagggtcaccatctcttgttctggaagtagctccaacatcggaacttatcctgtcaactggtaccagaagctcccaggaacggcccccaagctcctcatctatactaataatcagcggccctcaggggtccctgaccgattctctggctccaagtctggcacctcagcctccctgaccatcagtgggctccagtctgaggatgaggctgattattactgtgcaacatggggtgacagcctgaatgctgtggtattcggcggagggaccaaggtcaccgtccta

SEQ ID NO: 125 - DNA, 330 bp, Homo sapiens
  source 1..330: mol_type = other DNA
cagtctgccctgactcagcctgcctccgtgtctgggtctcctggacagtcgatcaccatctcctgcactggaaccagcagtgacgttggtggttataactatgtctcctggtaccaacagcacccaggcaaagcccccaaactcatgatttatgatgtcactaagcggccctcaggggtttctaatcgcttctctggctccaagtctggcaacacggcctccctgaccgtctctgggctccaggctgaggacgaggctgattattactgcagctcatatacaagcagtagaactttagtcttcggcggagggaccaaggtcaccgtccta

SEQ ID NO: 126 - DNA, 327 bp, Homo sapiens
  source 1..327: mol_type = genomic DNA
gatgtcgtgatgacccagactccagcctccgtgtctgaacctgtgggaggcacagtcaccatcaagtgccaggccagtcagaatattggtagtaatttagcctggtatcagcagaaaccagggcagcctcccaacctcctgatctatggtgcatccaatctggcatctggggtctcatcgcggttcaaaggcagtagctctgggacagagtacactctcaccatcagcgacctggagtgtgccgatgctgccacttactactgtcaatgtacttattatggtagtagttatgttgctttcggcggagggaccgaggtggtcgtcaaa

SEQ ID NO: 127 - DNA, 330 bp, Homo sapiens
  source 1..330: mol_type = other DNA
gcagccgtgctgacccagactacatcccccgtgtctggagctgtgggaggcacagtcaccatcaagtgccaggccagtcagagcgttggtagtaatttagcctggtatcagcagaaaccagggcagcctcccaagatcctgatcggtggtgcatccaaactggcatctggggtcccatcgcggttcagtggcagtggatctgggacagagttcactctcaccatcagcgacctggagtgtgccgatgctggcacttactactgtcacagttattattatagtagtactagtgctgatgctttcggcggagggaccaaggtggtcgtcgaa

SEQ ID NO: 128 - DNA, 330 bp, Homo sapiens
  source 1..330: mol_type = other DNA
gatgtcgtgatgacccagactacatcccccgtgtctggagctgtgggaggcacagtcaccatcaagtgccaggccagtcagagcgttggtactaatttagcctggtatcagcagaaaccagggcagcctcccaagatcctgatcggtggtgcatccaaactggcgtctggggtcccatcgcggttcagtggcagtggatctgggacagagttcactttcaccatcagcgacctggagtgtgccgatgctggcacttactactgtcacagttattattatagtagtactagtgctgatgctttcggcggagggaccgaggtggtcgtcaaa

SEQ ID NO: 129 - DNA, 339 bp, Homo sapiens
  source 1..339: mol_type = other DNA
gcccttgtgatgacccagactccagcctccgtggaggcagctgtgggaggcacagtcaccatcaagtgccaggccagtcagaacatttacagcaatttagcctggtatcagcagaaaccagggcagcctcccaagcgcctgatctacaaggcatccactctgccatctggggtcccatcgcggttcaaaggcagtggatctgggacacagttcactctcaccatcagcgacctggagtgtgccgatgctgccacttactactgtcaaagcaattattatagtagtagtagtaattatggccctaatgctttcggcggagggaccgaggtggtcgtcaaa

SEQ ID NO: 130 - DNA, 321 bp, Homo sapiens
  source 1..321: mol_type = other DNA
gatgtcgtgatgacccagactccagcctccgtgtctgaacctgtgggaggcacagtcaccatcaattgccaggccattcagagcattggtaataattttgtctggtatcagcagaaaccagggcagcctcccaagctcctgatctacaaggcatccattcgggcatctggggtctcatcgcggttcaggggcagtggatctgggacagacttcactctcaccatcagcgacctggagtgtgacgatactggcacttactactgtcaatgtacttatgctagtagtgctactttcggcggagggaccgaggtggtcgtcaaa

SEQ ID NO: 131 - DNA, 330 bp, Homo sapiens
  source 1..330: mol_type = other DNA
gaccctgtgctgacccagactccatcgtccgtgtctgcagctgtgggaggcacagtcaccatcagttgccaggccactgagactgtttatagtaacgaccgcttagcctggtatcagcagaaaccagggcagcctcccaagctcctgatctacaggatatccactctggaatctggggtcccatcgcggttcaaaggcagtggatctgggacacagttcactctcaccatcagcggcgtgcagtgtgacgatgctgccacttactactgtgcaggcggttataatggtaatatttgggctttcggcggagggaccgaggtggtcgtcaaa

SEQ ID NO: 132 - DNA, 333 bp, Homo sapiens
  source 1..333: mol_type = other DNA
gatgtcgtgatgacccagactgcatcccccgtgtctggagctgtgggaggcacagtcaccatcaagtgccaggccagtgaggacatttacagctctttagcctggtatcagcagaaaccagggcagcctcccaagctcctgatctaccaggcatccaaactggcatctggggtcccaccgcggttcagcggcagtggatctgggacagagtacactctcaccatcagcgacctggagtgtgccgatgctgccacttactactgtcagaactattattatgttcgtggtgctgattattgggctttcggcggagggaccgaggtggtcgtcgaa

(19) United States
(12) Patent Application Publication
(10) Pub. No.: US 2024/0376199 A1
(43) Pub. Date: Nov. 14, 2024

(54) TREM2 ANTIGEN BINDING PROTEINS AND USES THEREOF

(71) Applicant: THE BOARD OF REGENTS OF THE UNIVERSITY OF TEXAS SYSTEM, Austin, TX (US)

(72) Inventors: Zhiqiang AN, Houston, TX (US); Ningyan ZHANG, Houston, TX (US); Peng ZHAO, Houston, TX (US)

(21) Appl. No.: 18/691,229

(22) PCT Filed: Sep. 13, 2022
(86) PCT No.: PCT/US2022/076382

§ 371 Date: Mar. 12, 2024

Related U.S. Application Data
(60) Provisional application No. 63/243,431, filed on Sep. 13, 2021.
(60) Provisional application No. 63/321,235, filed on Mar. 18, 2022.

(51) Int. Cl. C07K 16/28

(52) U.S. Cl. CPC C07K 16/2803; C07K 16/2881; C07K 2317/31; C07K 2317/35; C07K 2317/565; C07K 2317/75

(57) ABSTRACT

The present disclosure is generally directed to compositions that include antibodies, e.g., monoclonal, antibodies, antibody fragments etc., that specifically bind a TREM2 protein or a TREM2 protein epitope, e.g., a mammalian TREM2 or human TREM2, and use of such compositions in preventing, reducing risk, or treating an individual in need thereof.

### REFERENCE TO A SEQUENCE LISTING SUBMITTED VIA ELECTRONIC FILING

The content of the XML file of the sequence listing named “UTH-012PCTO_sequence_listing_ST26_FILED.XML” which is 114 KB in size was created on Sep. 11, 2022, and electronically submitted to the USPTO's Patent Center herewith the present application is incorporated by reference in its entirety.

### CROSS-REFERENCE TO RELATED APPLICATIONS

This application claims the benefit of U.S. Provisional Appl. Nos. U.S. Provisional Application Ser. No. 63/243,431, filed Sep. 13, 2021, and entitled “TREM2 ANTIGEN BINDING PROTEINS AND USES THEREOF”, and to U.S. Provisional Application Ser. No. 63/321,235, filed Mar. 18, 2022, and entitled “TREM2 ANTIGEN BINDING PROTEINS AND USES THEREOF”. The content of the foregoing applications are relied upon and is incorporated by reference herein in its entirety.

### TECHNICAL FIELD

The present disclosure relates to anti-TREM2 antibodies and therapeutic uses of such antibodies.

### BACKGROUND

Alzheimer's disease (AD) is a neurodegenerative disease featuring beta-amyloid (Ab) deposition. The brain resident myeloid cells, microglia, are key in regulating AD pathology and controlling amyloid pathology. A Triggering Receptor Expressed on Myeloid cells (TREM2) has been discovered as a key regulator of microglia by controlling microglia activation and metabolic activities in brain tissues.

Variations in triggering receptor expressed on myeloid cells-2 (TREM2) have been shown to increase the risk of developing late-onset AD. Microglia have been shown to respond to Δβ accumulation and neurodegenerative lesions, progressively acquiring unique transcriptional and functional properties that eventually result in disease-associated microglia (DAM). DAM attenuate the progression of neurodegeneration in certain mouse models, but inappropriate DAM activation may accelerate neurodegenerative diseases. TREM2 is essential for maintaining microglial metabolic fitness during stress events, enabling microglial progression to a fully mature DAM profile and ultimately sustaining the microglial response to Aβ-plaque-induced pathology.

### SUMMARY

The present disclosure is generally directed to compositions that include antibodies, e.g., monoclonal, chimeric, humanized antibodies, antibody fragments, etc., that specifically bind a TREM2 protein, e.g., a mammalian TREM2 (e.g., any non-human mammal) or human TREM2, and to methods of using such compositions.

The inventors have created and characterized certain monoclonal antibodies with binding specificity to TREM2, a triggering receptor protein associated inter alia with microglial fitness during stress events and with microglial response to Ab-plaque-induced pathology. In addition, the investigators have created agonist monoclonal antibodies to TREM2 as well as antagonist monoclonal antibodies to TREM2. Surprisingly, said antagonistic monoclonal antibodies may be used for the treatment of cancer. The present disclosure provides polypeptides with affinity to TREM2, polynucleotides that encode the polypeptides, and methods of producing the polypeptides.

In one aspect, the present disclosure provides an isolated monoclonal antibody, or an antigen-binding fragment thereof, wherein the antibody specifically binds to TREM2 and wherein

In yet another aspect, the present disclosure provides a host cell comprising a polynucleotide molecule encoding a polypeptide of any one of the above embodiments.

Additional details of these and other embodiments are set forth in the accompanying drawings and the description below. Other features, objects and advantages will become apparent from the description and drawings, and from the accompanying claims.

An “agonist” antibody or an “activating” antibody is an antibody that induces (e.g., increases) one or more activities or functions of the antigen after the antibody binds the antigen.

An “antagonist” antibody or a “blocking” antibody is an antibody that reduces or eliminates (e.g., decreases) antigen binding to one or more ligand after the antibody binds the antigen, and/or that reduces or eliminates (e.g., decreases) one or more activities or functions of the antigen after the antibody binds the antigen. In some embodiments, antagonist antibodies, or blocking antibodies substantially or completely inhibit antigen binding to one or more ligand and/or one or more activities or functions of the antigen.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full-length of the sequences being compared.

An “isolated” nucleic acid molecule encoding an antibody, such as an anti-TREM2 antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acid encoding the polypeptides and antibodies herein existing naturally in cells.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

The term “comprising” and variations thereof (e.g., comprises, includes, etc.) do not have a limiting meaning where these terms appear in the description and claims.

As used herein, “a”, “an”, “the,” “at least one,” and “one or more” are used interchangeably, unless the context clearly dictates otherwise.

All scientific and technical terms used herein have meanings commonly used in the art unless otherwise specified. The definitions provided herein are to facilitate understanding of certain terms used frequently in this application and are not meant to exclude a reasonable interpretation of those terms in the context of the present disclosure.

It is understood that aspect and embodiments of the present disclosure described herein include “comprising,” “consisting,” and “consisting essentially of” aspects and embodiments.

The above summary of the present invention is not intended to describe each disclosed embodiment or every implementation of the present invention. The description that follows more particularly exemplifies illustrative embodiments.

### BRIEF DESCRIPTION OF DRAWINGS

FIG. 1A-H. Screening and characterization of TREM2 agonist AbM8. a. Illustration of TREM2 antibody screening strategy starting from TREM2-binding of scFv-displayed on phage; b. Candidate antibodies binding to cell surface TREM2 expressed on HEK293T cells by flow cytometry. MFI: mean fluorescence intensity, n=3 independent repeats; c. Ligand competition profiles of antibody candidates (10 nM, n=3 independent repeats) on TREM2-DAP12 NFAT-EGFP reporter cells; d. Titration curves of antibody candidates in activating TREM2-DAP12 NFAT-EGFP reporter cells. The EC₅₀ of Ab18 is labeled next to its titration curve. n=3 independent repeats; e. Titration curves of Ab18 for stimulating oAβ-lipid phagocytosis in mouse neonatal microglia. MFI: mean fluorescence intensity. The EC₅₀ of Ab18 is labeled next to its titration curve. n=3 independent repeats; f. representative immunofluorescence imaging showing Ab18 stimulates oAβ-lipid phagocytosis in mouse neonatal microglia. Scale bar=10 μm; g. representative immunofluorescence imaging showing Ab18 binding TREM2 express on 293T surface, Scale bar=20 μm; and h. representative immunofluorescence imaging showing Ab18 binding mouse neonatal microglia, Scale bar=20 μm. For all the data displayed, a bar graph with error bars represents mean±SD.

FIG. 2A-I. Antibody engineering of TREM2 agonist Ab18 and characterization of in vitro biological effects. a. illustration shows the 6 antibody formats used in the format engineering study of Ab18. Although not depicted, all antibodies bear the LALAPG mutations; b. Titration curves of various Ab18 format candidates in activating TREM2-DAP12 NFAT-EGFP reporter cells. The EC₅₀ of Ab18 TVD-Ig is labeled next to its titration curve. n=3 independent repeats; c. Immunoblot data showing the increased phosphorylation of SYK (as pSYK) of mouse neonatal microglia treated by antibodies; d. Titration curves of antibody-mediated SYK phosphorylation in mouse neonatal microglia. Y-axis plots the fold change normalized to β-actin and the untreated cell control. n=3 independent repeats; e. Titration curves of antibody-mediated stimulation of oAβ-lipid phagocytosis in mouse neonatal microglia. MFI: mean fluorescence intensity. n=3 independent repeats; f. representative immunofluorescence imaging showing Ab18 TVD-Ig (10 nM) stimulates oAβ-lipid phagocytosis in mouse neonatal microglia, which Ab18 IgG fails. Scale bar=10 μm; g. representative immunofluorescence imaging showing Ab18 TVD-Ig (10 nM) stimulates migration of mouse neonatal microglia toward oAβ-lipid, which Ab18 IgG fails. Scale bar=70 μm; h. Titration curves of antibody-mediated stimulation of mouse neonatal microglia migration toward oAβ-lipid. Y-axis presents the percentage of migration, which was calculated based on the migrated cell number divided by the total cell number. n=3 independent repeats; i. Titration curves of antibody-mediated stimulation of mouse neonatal microglia survival under low CSF1 concentration (5 ng/mL). Y-axis presents percentage luminescence values produced by the ATP from live cells. n=3 independent repeats. For all the data displayed, a bar graph with error bars represents mean±SD.

FIG. 3A-H. Molecular mechanisms of Ab18 TVD-Ig for enhanced TREM2 activation. a. SEC profiles showing Ab18 TVD-Ig induce TREM2 clustering with the size of complexes significantly larger than the one formed between Ab18 IgG and TREM2; b. elution volume of the antibody-TREM2 complexes formed in a. Smaller elution volume means larger molecular weight (size); c. representative immunofluorescence imaging showing Ab18 TVD-Ig (10 nM) stimulates TREM2 clustering in mouse neonatal microglia, while Ab18 IgG only shows cell surface staining. Scale bar=20 μm; d. Flow cytometry results of TREM2 cell surface level in antibody-treated neonatal mouse microglia. MFI: mean fluorescence intensity. The Figure legend labels the antibody treatment and the staining marker. n=3 independent repeats; e. representative immunoblot data showing sTREM2 level in the supernatant of antibody-treated mouse neonatal microglia; f. quantification of the immunoblot data in e. Y-axis plots the fold change normalized to APP and the untreated cell control. n=3 independent repeats; g. representative immunoblot data showing total TREM2 level in the antibody-treated mouse neonatal microglia; h. quantification of the immunoblot data in g. Y-axis plots the fold change normalized to calnexin and the untreated cell control. n=3 independent repeats. For all the data displayed, a bar graph with error bars represents mean±SD. For the statistical analysis, ns=not statistically different, two-tailed Student t-test.

FIG. 4A-G. Ab18/αTfR bispecific antibody characterization. a. illustration showing the design of Ab18/αTfR and the corresponding control bispecific antibodies; b. sandwich BLI study showing the single-molecule incorporation of both Ab18 and αTfR. The corresponding proteins involved in each curve were labeled above the dotted lines indicating the start of each binding phase. “Wash” means dipping the sensor into a blank kinetics buffer to allow free dissociation; c. Titration curves of TREM2-DAP12 NFAT-EGFP reporter cells treated by antibodies. The EGFP+ percentage was measured by flow cytometry. n=3 independent repeats; d. antibody concentrations in perfused brains at designated time points after treatment (20 mg/kg through intraperitoneal injection). n=5 independent mice; e. antibody concentrations in serum at designated time points after treatment as described in d. n=5 independent mice; f. brain/serum antibody concentration ratios at designated time points after treatment as described in d. The ratios presented in Y-axis were calculated by brain antibody concentration (nM) divided by serum antibody concentration (μM) times 100. n=5 independent mice; g. representative immunofluorescence imaging showing the antibody distribution in mouse brains 24 hours after treatment (20 mg/kg through intraperitoneal injection). To detect Ab18 distribution, an anti-human secondary antibody was used. The same brain slices were also co-stained with CD31 (blood vessel), IBA1 (microglia), and TO-PRO-3 (nucleus) to allow co-localization visualization. Scale bar=20 μm. For all the data displayed, a bar graph with error bars represents mean±SD.

FIG. 5A-D. Ab18/αTfR ameliorate amyloid pathology in 5×FAD mice. a. representative amyloid plaque immunofluorescence staining of the brain slices of 5×FAD mouse treated by designated antibodies (20 mg/kg, 14 weekly intraperitoneal dosages started when mice were 5-mo-old). Scale bar=70 μm; b. overall 6E10 staining intensity quantification of mice treated as described in a. n=5 independent mice; c. amyloid plaque burden in the cortex of mice treated as described in a. n=5 independent mice; d. amyloid plaque burden in the hippocampus of mice treated as described in a. n=5 independent mice. For all the data displayed, a bar graph with error bars represents mean±SD. For the statistical analysis, ns=not statistically different, ***P<0.001, two-tailed Student t-test.

FIG. 6A-F. Impact of Ab18/αTfR in microglia and astrocyte responses to amyloid plaques. a. representative amyloid plaque-microglia co-localization immunofluorescence staining of the cortex of 5×FAD mouse treated by designated antibodies (20 mg/kg, 14 weekly intraperitoneal dosages started when mice were 5-mo-old). Scale bar=20 μm; b. representative amyloid plaque-CD68 co-localization immunofluorescence staining of the cortex of 5×FAD mice treated as described in a. Scale bar=20 μm; c. representative amyloid plaque-GFAP co-localization immunofluorescence staining of the cortex of 5×FAD mice treated as described in a. Scale bar=20 μm; d. quantification of IBA1 area within 30 μm of amyloid plaques in the brain slices of mice treated as described in a. n=5 independent mice; e. quantification of CD68 area within 30 μm of amyloid plaques in the brain slices of mice treated as described in a. n=5 independent mice; f. quantification of GFAP area within 30 μm of amyloid plaques in the brain slices of mice treated as described in a. n=5 independent mice. For all the data displayed, a bar graph with error bars represents mean f SD. For the statistical analysis, ns=not statistically different, ***P<0.001, two-tailed Student t-test.

FIG. 7A-D. Ab18/αTfR ameliorates neuronal damage without overall neuron density loss. a. representative amyloid plaque-LAMP1 co-localization immunofluorescence staining of the cortex of 5×FAD mouse treated by designated antibodies (20 mg/kg, 14 weekly intraperitoneal dosages started when mice were 5-mo-old). Scale bar=70 μm; b. quantification of LAMP1 area within 30 μm of amyloid plaques in the brain slices of mice treated as described in a. n=5 independent mice; c. representative NeuN immunofluorescence staining of the brain slices of 5×FAD mice treated as described in a. Scale bar=70 μm; d. quantification of NeuN intensity in the brain slices of mice treated as described in a. n=5 independent mice. For all the data displayed, a bar graph with error bars represents mean*SD. For the statistical analysis, ns=not statistically different, ***P<0.001, two-tailed Student t-test.

### DETAILED DESCRIPTION

All publications mentioned herein are incorporated by reference to the extent they support the present invention.

The present disclosure describes monoclonal antibodies, and fragments thereof, with binding affinity to TREM2.

Anti-TREM2 antibodies are disclosed in US Patent Publication No. US20190040130A1 and PCT Patent Publication No. WO2018195506A1, each of which is incorporated herein in its entirety. In contrast to the methods used to produce the antibodies in those publications, the present disclosure describes some antibodies produced by panning a bacteriophage display library to identify clones with TREM2 binding affinity. While other monoclonal antibodies of the present disclosure were created by making hybridomas using B-cells from immunized rabbits. These monoclonal antibodies were subsequently humanized using techniques that are known in the art.

### Antibodies

In certain embodiments, an antibody or a fragment thereof that binds to at least a portion of TREM2 protein and modulates (e.g., activates, increases, decreases, or blocks) at least one microglia function is contemplated. As used herein, the term “antibody” is intended to refer broadly to any immunologic binding agent, such as IgG, IgM, IgA, IgD, IgE, and genetically modified IgG as well as polypeptides comprising antibody CDR domains that retain antigen binding activity. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. In some embodiments, the TREM2-binding antibody is a monoclonal antibody or a humanized antibody.

An “antibody molecule” encompasses an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), for example IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen binding portion” of an antibody molecule, as used herein, refers to one or more fragments of an intact antibody that retain the ability to specifically bind to the target molecule (e.g., TREM2). Antigen binding functions of an antibody molecule can be performed by fragments of an intact antibody. Examples of binding fragments encompassed within the term “antigen binding portion” of an antibody molecule include Fab; Fab′; F(ab′)2; an Fd fragment consisting of the V_{H} and CH1 domains; an Fv fragment consisting of the V_{L} and V_{H} domains of a single arm of an antibody; a single domain antibody (dAb) fragment, and an isolated complementarity determining region (CDR).

The term “Fc region” is used to define a C-terminal region of an immunoglobulin heavy chain. The “Fc region” may be a native sequence Fc region or a variant Fc region. Although the boundaries of the Fc region of an immunoglobulin heavy chain might vary, the human IgG heavy chain Fc region is usually defined to stretch from an amino acid residue at position Cys226, or from Pro230, to the carboxyl-terminus thereof. The numbering of the residues in the Fc region is that of the EU index as in Kabat. The Fc region of an immunoglobulin generally comprises two constant domains, CH2 and CH3. As is known in the art, an Fc region can be present in dimer or monomeric form.

A “variable region” of an antibody refers to the variable region of the antibody light chain or the variable region of the antibody heavy chain, either alone or in combination. As known in the art, the variable regions of the heavy and light chain each consist of four framework regions (FRs) connected by three complementarity determining regions (CDRs) also known as hypervariable regions, contribute to the formation of the antigen binding site of antibodies. When choosing FR to flank CDRs, for example when humanizing or optimizing an antibody, FRs from antibodies which contain CDR sequences in the same canonical class are preferred.

As used herein the term “conservative substitution” refers to replacement of an amino acid with another amino acid which does not significantly deleteriously change the functional activity. A preferred example of a “conservative substitution” is the replacement of one amino acid with another amino acid (see for example, Henikoff & Henikoff, 1992, PNAS 89: 10915-10919).

Thus, by known means and as described herein, monoclonal antibodies, antibody fragments, and binding domains and CDRs (including engineered forms of any of the foregoing) may be created that are specific to TREM2 protein, one or more of its respective epitopes, or conjugates of any of the foregoing, whether such antigens or epitopes are isolated from natural sources or are synthetic derivatives or variants of the natural compounds.

Examples of antibody fragments suitable for the present embodiments include, without limitation: (i) the Fab fragment, consisting of V_{L}, V_{H}, CL, and CHI domains; (ii) the “Fd” fragment consisting of the V_{H} and CHI domains; (iii) the “Fv” fragment consisting of the V_{L} and V_{H} domains of a single antibody; (iv) the “dAb” fragment, which consists of a V_{H} domain; (v) isolated CDR regions; (vi) F(ab′)2 fragments, a bivalent fragment comprising two linked Fab fragments; (vii) single chain Fv molecules (“scFv”), wherein a V_{H} domain and a V_{L} domain are linked by a peptide linker that allows the two domains to associate to form a binding domain; (viii) bi-specific single chain Fv dimers (see, for example, U.S. Pat. No. 5,091,513); and (ix) diabodies, multivalent or multispecific fragments constructed by gene fusion (see, for example, US Patent App. Pub. No. 20050214860, which is incorporated herein by reference in its entirety). Fv, scFv, or diabody molecules may be stabilized by the incorporation of disulphide bridges linking the V_{H} and V_{L} domains. Minibodies comprising a scFv joined to a CH3 domain may also be made (See, for example, Hu et al, 1996, “Minibody: A Novel Engineered Anti-Carcinoembryonic Antigen Antibody Fragment (Single-Chain Fv-CH3) Which Exhibits Rapid, High-Level Targeting of Xenografts”, Cancer Res. 56:3055-3061, which is incorporated herein by reference in its entirety).

Antibody-like binding peptidomimetics are also contemplated in embodiments. Liu et al. (Murali, R.; Liu, Q.; Cheng, X.; Berezov. A.; Richter, M.; Furuchi, K.; Greene, M. I.; Zhang, H. Antibody like peptidomimetics as large scale immunodetection probes. Cell. Mol. Biol. (Noisy-le-grand) 2003, 49:209-216, which is incorporated herein by reference in its entirety) describe “antibody like binding peptidomimetics” (ABiPs), which are peptides that act as pared-down antibodies and have certain advantages of longer serum half-life as well as less cumbersome synthesis methods.

A monoclonal antibody (or “MAb”) is a single species of antibody wherein every antibody molecule recognizes the same epitope because all antibody producing cells are derived from a single B-lymphocyte cell line. The methods for generating monoclonal antibodies (MAbs) generally begin along the same lines as those for preparing polyclonal antibodies. In some embodiments, rodents such as mice and rats are used in generating monoclonal antibodies. In some embodiments, rabbit, sheep, or frog cells are used in generating monoclonal antibodies. The use of rats is well known and may provide certain advantages. Mice (e.g., BALB/c mice) are routinely used and generally give a high percentage of stable fusions.

Hybridoma technology involves the fusion of a single B lymphocyte from a mouse previously immunized with a TREM2 antigen with an immortal myeloma cell (usually mouse myeloma). This technology provides a method to propagate a single antibody producing cell for an indefinite number of generations, such that unlimited quantities of structurally identical antibodies having the same antigen or epitope specificity (monoclonal antibodies) may be produced.

Plasma B cells (CD45+CD5−CD19+) may be isolated from freshly prepared rabbit peripheral blood mononuclear cells of immunized rabbits and further selected for TREM2 binding cells. After enrichment of antibody producing B cells, total RNA may be isolated and cDNA synthesized. DNA sequences of antibody variable regions from both heavy chains and light chains may be amplified, constructed into a phage display Fab expression vector, and transformed into E. coli. TREM2 specific binding Fab may be selected out through multiple rounds enrichment panning and sequenced. Selected TREM2 binding hits may be expressed as full length IgG in rabbit and rabbit/human chimeric forms using a mammalian expression vector system in human embryonic kidney (HEK293) cells (Invitrogen) and purified using a protein G resin with a fast protein liquid chromatography (FPLC) separation unit.

In one embodiment, the antibody is a chimeric antibody, for example, an antibody comprising antigen binding sequences from a non-human donor grafted to a heterologous non-human, human, or humanized sequence (e.g., framework and/or constant domain sequences). Methods have been developed to replace light and heavy chain constant domains of the monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see, for example, U.S. Pat. Nos. 5,091,513 and 6,881,557, which are incorporated herein by reference in their entirety). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications, which are incorporated herein by reference in their entirety, provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024.

Antibodies may be produced from any animal source, including birds and mammals. Preferably, the antibodies are ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference.

Without being bound by theory, it is believed that antibodies to TREM2 will have the ability to modulate, by binding to TREM2, human microglia activity regardless of the source (e.g., animal species, monoclonal cell line, or other source) of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into antibody fragments having the binding domain or CDR. Removal of the Fc portion reduces the likelihood that the antigen antibody fragment will elicit an undesirable immunological response, and thus, antibodies without Fc may be preferential for prophylactic or therapeutic treatments. As described above, antibodies may also be constructed so as to be chimeric or partially or fully human, so as to reduce or eliminate the adverse immunological consequences resulting from administering to an animal an antibody that has been produced in, or has sequences from, other species.

It is contemplated that substitutional variants may contain the exchange of one amino acid for another at one or more sites within the monoclonal antibody protein and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins (e.g., monoclonal antibodies) of the present disclosure may be isolated (e.g., enriched and/or purified to some degree) and/or may be recombinant or synthesized in vitro. Alternatively, a nonrecombinant or recombinant protein may be isolated from bacteria. It is also contemplated that a bacteria containing such a variant may be implemented in compositions and methods. Consequently, a protein need not be isolated.

Thus, the present disclosure provides an isolated or recombinant monoclonal antibody that specifically binds to TREM2. In certain aspects, an antibody that competes for the binding of TREM2 with the TREM2-Ab2Hu, TREM2-Ab8Hu, TREM2-Ab19Hu, TREM2-Ab1Rb, TREM2-Ab2Rb, TREM2-Ab6Rb, TREM2-Ab12Rb, TREM2-Ab16Rb, TREM2-Ab22Rb, or TREM2-Ab26Rb monoclonal antibody (each disclosed and described herein) is provided. In certain aspects, the antibody may comprise all or part of the heavy chain variable region and/or light chain variable region of the TREM2-Ab2Hu, TREM2-Ab8Hu, TREM2-Ab19Hu, TREM2-Ab1Rb, TREM2-Ab2Rb, TREM2-Ab6Rb, TREM2-Ab12Rb, TREM2-Ab16Rb, TREM2-Ab22Rb, or TREM2-Ab26Rb monoclonal antibodies.

An antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

Embodiments provide antibodies and antibody-like molecules against TREM2, polypeptides and peptides that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3a-6a-diphenylglycouril attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

In another aspect, the present disclosure provides polynucleotides that can be expressed (e.g., transcribed and translated) in a suitable host to produce a TREM2-binding polypeptides or portions thereof. It is contemplated that such polynucleotide sequences can be cloned in a suitable expression vector by means known in the art and the expression vector can be used in vivo or in vitro to express the TREM2-binding polypeptide encoded by the polynucleotide sequences.

In certain embodiments, a polynucleotide of the present disclosure comprises a portion having at least 80%, 85%, 90%, 91%, 92%, 93%, 94%, 95%, 96%, 97%, 98%, 99%, or 100% sequence identity to H2-Hu-HC-DNA (SEQ ID NO: 134). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to H8-Hu-HC-DNA (SEQ ID NO: 135). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 19H-Hu HC-DNA (SEQ ID NO: 136). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 16H-HC-DNA (SEQ ID NO: 137). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 22H-HC-DNA (SEQ ID NO: 138). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 26H-HC-DNA (SEQ ID NO: 139). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to H2-Hu-HC-DNA (SEQ ID NO: 140). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to H8-Hu-HC-DNA (SEQ ID NO: 141). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to H19-Hu-HC-DNA (SEQ ID NO: 142).

In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 20L-LC-DNA (SEQ ID NO: 143). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 8L-LC-DNA (SEQ ID NO: 144). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 19L LC-DNA (SEQ ID NO: 145). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 1K-LC-DNA (SEQ ID NO: 146). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 2K-LC-DNA (SEQ ID NO: 147). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 6K-LC-DNA (SEQ ID NO: 148). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 12K-LC-DNA (SEQ ID NO: 149). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 16K-LC-DNA (SEQ ID NO: 150). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 22K-LC-DNA (SEQ ID NO: 151). In certain embodiments, a polynucleotide of the present disclosure comprises a portion having at least 80%, 85%, 90%, 91%, 92%, 93%, 94%, 95%, 96%, 97%, 98%, 99%, or 100% sequence identity to 26K-LC-DNA (SEQ ID NO: 152).

### Treatment of Diseases

Certain aspects of the present embodiments can be used to prevent or treat a disease or disorder associated with TREM2-regulated proteins (e.g., diseases of the brain associated with beta-amyloid peptides and other such neurodegenerative diseases and disorders including, but, not limited to Alzheimer's Disease (AD), Parkinson's Disease (PD), dementia, dementia with Lewy bodies (DLB) and others, including neuroinflammatory processes and those involving microglia, for example). TREM2 activity may be increased or reduced by any TREM2-binding antibodies.

“Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an antibody that modulates TREM2 biological activity.

“Subject” and “patient” refer to either a human or non-human, such as primates, mammals, and vertebrates. In particular embodiments, the subject is a human.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

### Pharmaceutical Preparations

Where clinical application of a therapeutic composition containing an antibody is undertaken, it will generally be beneficial to prepare a pharmaceutical or therapeutic composition appropriate for the intended application. In certain embodiments, pharmaceutical compositions may comprise, for example, at least about 0.1% of an active compound. In other embodiments, an active compound may comprise between about 2% to about 75% of the weight of the unit, or between about 25% to about 60/a, for example, and any range derivable therein.

The therapeutic compositions of the present embodiments are advantageously administered in the form of injectable compositions either as liquid solutions or suspensions; solid forms suitable for solution in, or suspension in, liquid prior to injection may also be prepared. These preparations also may be emulsified.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “unit dose” or “dosage” refers to physically discrete units suitable for use in a subject, each unit containing a predetermined quantity of the therapeutic composition calculated to produce the desired responses discussed above in association with its administration, i.e., the appropriate route and treatment regimen. The quantity to be administered, both according to number of treatments and unit dose, depends on the effect desired. The actual dosage amount of a composition of the present embodiments administered to a patient or subject can be determined by physical and physiological factors, such as body weight, the age, health, and sex of the subject, the type of disease being treated, the extent of disease penetration, previous or concurrent therapeutic interventions, idiopathy of the patient, the route of administration, and the potency, stability, and toxicity of the particular therapeutic substance. For example, a dose may also comprise from about 1 mg/kg/body weight to about 1000 mg/kg/body weight (this such range includes intervening doses) or more per administration, and any range derivable therein. In non-limiting examples of a derivable range from the numbers listed herein, a range of about 5 mg/kg/body weight to about 100 mg/kg/body weight, about 5 mg/kg/body weight to about 500 mg/kg/body weight, etc., can be administered. The practitioner responsible for administration will, in any event, determine the concentration of active ingredient(s) in a composition and appropriate dose(s) for the individual subject.

The active compounds can be formulated for parenteral administration, e.g., formulated for injection via the intravenous, intramuscular, intrathecal, sub-cutaneous, or even intraperitoneal routes. Typically, such compositions can be prepared as either liquid solutions or suspensions; solid forms suitable for use to prepare solutions or suspensions upon the addition of a liquid prior to injection can also be prepared; and the preparations can also be emulsified.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

### Kits and Diagnostics

In various aspects of the embodiments, a kit is envisioned containing therapeutic agents and/or other therapeutic and delivery agents. In some embodiments, the present embodiments contemplate a kit for preparing and/or administering a therapy of the embodiments. The kit may comprise one or more sealed vials containing any of the pharmaceutical compositions of the present embodiments. The kit may include, for example, at least one anti-TREM-2 antibody as well as reagents to prepare, formulate, and/or administer the components of the embodiments or perform one or more steps of the inventive methods. In some embodiments, the kit may also comprise a suitable container, which is a container that will not react with components of the kit, such as an Eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### EXAMPLES

Unless noted otherwise, data generated from the experiments and Examples described herein below can be found in Zhao et al., Sci. Transl. Med. 14, eabq0095 (2022), which is incorporated herein by reference in its entirety.) Methods

### Cell Lines.

HEK293T was acquired from the American Type Culture Collection and cultured in DMEM+10% FBS. The 2B4 nuclear factor of activated T-cells (NFAT)-GFP reporter cell line was cultured in RPMI-1640+10% FBS.

Panning of Phage-Displayed scFv Antibody Library.

A phage-displayed scFv antibody library was prepared previously (Zhao, S., et al., Partial Leptin Reduction as an Insulin Sensitization and Weight Loss Strategy. Cell Metab, 2019.30(4): p. 706-719.e6). Panning of the library for TREM2 specific antibodies was carried out as described previously with modifications (Zhao, S., et al., Partial Leptin Reduction as an Insulin Sensitization and Weight Loss Strategy. Cell Metab, 2019. 30(4): p. 706-719.e6). Briefly, MaxiSorp Nunc-Immuno tubes (Thermo Fisher Scientific) were coated with 20 μg/mL mouse TREM2-His in DPBS overnight at 4° C. Unbound antigen was removed after washing with DPBS. After blocking the surface with 5% milk in DPBS, the phage library was incubated with the coated TREM2 for 2 hours at room temperature in 5% milk. After washing with PBS+0.05% tween-20 to remove unbound phage, captured phage was eluted by incubating with 100 mM TEA for 20 min. Eluted phage-infected log-phase growing E. coli TG1 were then amplified on 2×YTAG agar 500 cm² square plate (Corning) at 30° C. overnight. The amplified phage-infected TG1 cells were used to prepare the phage for the next round of panning using the M13KO7 helper phage. The enrichment process was performed for three rounds using the output from the previous round as the input for the next round. After three rounds of panning, the output titer was measured and single colonies were used to prepare phage for ELISA. High-binding ELISA plates (Corning) were coated with TREM2-His at 2 μg/mL overnight at 4° C. After blocking with 5% milk in PBS, phage prepared from single TG1 colonies in 5% milk PBS was incubated with coated TREM2 for 1 hour at room temperature. After washing with PBS+0.05% Tween-20, anti-M13-HRP (Santa Cruz Biotechnology) was added at 1:2000 concentration and incubated for 1 hour at room temperature. After washing with PBS+0.05% Tween-20, TMB substrate (Termo Fisher Scientific) was added and incubated for 5 min before being stopped by 1N H₂SO₄. OD values were read at 450 nm. Top 20% high-binding clones were selected. Phagemids were extracted using Qiagen BioRobot Universal System in 96-well format. After DNA sequencing, sequences were analyzed using the IMGT V-quest service to identify antibody sequences with unique CDR3 regions.

### Construction and Production of Antibodies.

Unique scFv clones were converted into human IgG1 using mixed universal primers with degeneracy (Zhao, S., et al., Partial Leptin Reduction as an Insulin Sensitization and Weight Loss Strategy. Cell Metab, 2019. 30(4): p. 706-719.e6). Panning of the library for TREM2 specific antibodies was carried out as described previously with modifications (Zhao, S., et al., Partial Leptin Reduction as an Insulin Sensitization and Weight Loss Strategy. Cell Metab, 2019. 30(4): p. 706-719.e6). Individual heavy and light variable chains were amplified using PrimeStar GXL polymerase (Takara Bio). Gel-purified variable chain fragments were cloned into digested vectors using In-fusion HD cloning enzyme mix (Takara Bio). After the converted plasmid was sequenced, sequences verified IgG plasmids were transfected into Expi293F cells at the 2-mL scale. The ratios between the heavy chain and light plasmids are 1:1. After culturing for 5 days, cells were removed and antibody-containing supernatant was collected for screening assay.

For constructing various antibody formats and bispecific antibodies, the corresponding gene fragments were fused as follows. The desired gene fragments were first amplified using PrimeStar GXL polymerase (Takara Bio); and up to 3 fragments were then fused to create the whole or part of the novel antibody format using In-fusion HD cloning enzyme mix (Takara Bio) until the desired constructs were made. When expressing in Expi293F, the heavy and light chain plasmids were co-transfected at equal weight ratios. For milligram-scale antibody purification, Expi293F-produced antibodies were purified using CaptivA Protein-A affinity resin (Repligen) and eluted with 0.1M glycine (pH=2.5) and then neutralized with 1/20 volume 1M Tris-HCl (pH=9). Buffer exchange to DPBS was done using Amicon Ultra-15 ultrafiltration units (Mw cutoff=30 k) (MilliporeSigma).

### Cell Immunofluorescence Assays.

Cells were seeded in 8-well chamber slides (hermo Scientific) at indicated density. For Expi293T and Expi293T-TREM2, the density is 4×10⁴ cells per well. For microglia, the density is 5×10⁴ cells per well. For microglia phagocytosis, 1 μM oAβ-lipid (Alexa Fluor 555 labeled) was mixed with indicated antibodies and incubated with overnight cultured cells for 2 hours in 1% BSA PBS. After the phagocytosis experiment, cells were fixed 15 min in 4% PFA at 4° C. The nucleus was labeled with TO-PRO-3 (Thermo Scientific) at 1 μM for 15 min at RT. Cells were then mounted using ProLong Gold Antifade Mountant (Thermo Scientific) and imaged using Leica TCS SP5 confocal microscope.

For Expi293T and Expi293T-TREM2 surface staining, overnight cultured cells were washed once with DPBS to remove the medium and then blocked in 1% BSA PBS for 1 hour. After fixing 15 min in 4% PFA at 4° C., the cells were washed once by DPBS to remove PFA. Ab18 (100 nM) was added in 1% BSA PBS for 1 hour, and excessive Ab18 was then washed away by DPBS 3 times. Anti-human Alexa Fluor 488 (Jackson Immunoresearch) was added at 1 μg/mL for 1 hour in 1% BSA PBS. The nucleus was labeled with TO-PRO-3 (Thermo Scientific) at 1 μM for 15 min at RT. Cells were then mounted using ProLong Gold Antifade Mountant (Thermo Scientific) and imaged using Leica TCS SP5 confocal microscope.

For microglia cell antibody staining, the procedure is similar to the Expi293T staining procedure described above, except biotinylated Ab18 (homemade using Sulfo-NHS-Biotin) was used and detected by streptavidin-Alexa Fluor 488 (Jackson Immunoresearch). During the entire blocking and incubation, 0.1 mg/mL human IgG1 Fc fragment (Jackson Immunoresearch) was added together with 1% BSA PBS to block interactions with Fc receptors,

### Microglia Viability

Mouse neonatal microglia were prepared as previously described (Xiang, X., et al., TREM2 deficiency reduces the efficacy of immunotherapeutic amyloid clearance. EMBO Mol Med, 2016. 8(9): p. 992-1004). After differentiation for 7 days, cells were washed and resuspended in culture media with designated antibodies and 5 ng/ml colony-stimulating factor (CSF)(Biolegend). After 5 days, cellular ATP levels were measured by luminescence detection to indicate cell viability with CellTiter-Glo (Promega).

### Size-Exclusion Chromatography (SEC).

SEC profiles of TREM2 antibody and TREM2 complexes were determined by AKTA pure protein purification system (Cytiva). Briefly, purified antibodies and mouse TREM2-His (Sino Biological) were mixed at a 2:1 ratio with the antibody at 1 mg/mL concentration. A total of 100 μl mixtures were injected. The analysis process run 36 mL PBS at 0.5 ml/min using isocratic gradient over a Superose 6 Increase 10/300 GL column in 1×PBS, pH 7.4 running buffer.

### Immunostaining of Mouse Brain Sections.

Brains were collected with half flash-frozen in liquid nitrogen and another half prepared for cryo-sectioning. For immunofluorescence, the half mouse brains were dipped into 4% PFA for 1 d, then 30% sucrose for 2 d before being embedded into OCT medium (Sakura) and sectioned using Leica Cryostat CM1950 into 40 μm floating coronal sections. The floating sections were stored at 4° C. in PBS with 0.01% sodium azide until use.

Floating sections were first blocked in 1% BSA PBS with 0.3% Triton X-100 for 2 hours, then stained with corresponding antibodies: CD31 (1:500, R&D system), streptavidin-Alexa Fluor 488 (1:500, Jackson Immunoresearch), ionized calcium-binding adaptor molecule 1 (IBA1) (1:1000, Wako), 6E10 (1:500, Biolegend), CD68 (1:500 Biolegend), glial fibrillary acidic protein (GFAP) (1:100, Santa Cruz Bio), lysosomal associated membrane protein 1 (LAMP1) (1:500, Biolegend), and neuronal nuclei antigen (NeuN) (1:1000, Biolegend), in 1% BSA PBS with 0.3% Triton X-100 for overnight at 4° C. with gentle rocking. After washing in PBS 0.3% Triton X-100, corresponding secondary antibodies with fluorescent labeling were incubated with brain slices for 2 hours at 4° C. with gentle rocking. The nucleus was stained with TO-PRO-3 (1 μM) in DPBS for 30 min and then mounted using ProLong Gold Antifade Mountant (Thermo Scientific). Brains slices were imaged using a Leica TCS SP5 confocal microscope. The quantification was done using ImageJ as previously described (Shihan, M. H., et al., A simple method for quantitating confocal fluorescent images. Biochem Biophys Rep, 2021. 25: p. 100916; Ghosh, A., et al., An epoxide hydrolase inhibitor reduces neuroinflammation in a mouse model of Alzheimer's disease. Sci Transl Med, 2020. 12(573)). For quantification of the fluorescent intensity of indicated markers in the mouse cortex and hippocampus, images were analyzed by ImageJ, and background was subtracted by the software for fluorescence images before quantification.

### Bispecific Antibody Validation by Bio-Layer Interferometry (BLI) Assay.

Streptavidin sensors (Fortebio) were used to capture biotinylated TREM2 proteins (Sino Biological). During all incubation steps, samples were kept at room temperature with 1000 rpm shaking. In the TREM2 loading step, 100 nM biotinylated TREM2 proteins were incubated with the sensors for the designated time. In the bispecific antibody interaction steps, 200 nM antibodies were used. In the muTfR incubation step, 100 nM muTfR-His (Sino Biological) were used. Between incubations, the sensors were dipped into blank kinetic buffers to allow the free dissociation of proteins.

### Immunoblotting

Cell lysate or brain lysate were obtained by lysing cells or brain tissues using NP-40 lysis buffer (1% NP40, 50 mM Tris-HCl, pH=8, 150 mM NaCl) with Halt™ Protease and Phosphatase Inhibitor Cocktail (100×) (Thermo Fisher) for 1 hour with rocking. After removing debris by centrifugation at 14,000 rpm for 10 min, the total protein amount was normalized by Pierce BCA Protein Assay Kit (Thermo Fisher). Protein samples were resolved by 10% SDS-polyacrylamide gels (Bio-Rad) and later transferred onto Immun-Blot PVDF membranes (Bio-Rad). Proteins were probed with specific primary antibodies and secondary antibodies diluted in 5% BSA TBST (Zhao, Y., et al., TREM2Is a Receptor for β-Amyloid that Mediates Microglial Function. Neuron, 2018. 97(5): p. 1023-1031.e7; Zhong, L., et al., Amyloid-beta modulates microglial responses by binding to the triggering receptor expressed on myeloid cells 2 (TREM2). Mol Neurodegener, 2018. 13(1): p. 15; Chen, H.-M., et al., Blocking immunoinhibitory receptor LILRB2 reprograms tumor-associated myeloid cells and promotes antitumor immunity. The Journal of Clinical Investigation, 2018. 128(12): p. 5647-5662). Antibodies used were SYK (1:1000, Cell Signaling Technology), phosphorylated spleen tyrosine kinase (pSYK) (1:1000, Cell Signaling Technology), ACTB (1:1000, Cell Signaling Technology), APP (1:500 Millipore Sigma), sTREM2, and TREM2 (1:500 Millipore Sigma), and Calnexin (1:1000, Abcam). The immunoreactive bands were visualized with the West Pico PLUS Chemiluminescent Substrate (Thermo Fisher). The immunoreactive bands were quantified using ImageJ. Three independent treatment replicates were conducted with the representative immunoblot shown.

### Antibody Brain Distribution Study.

The animal experiments were conducted according to the institutional guidelines with approved protocols. C57BL6 mice (female, 8-week-old, Jackson Laboratory) were randomly grouped into 5 mice per group. Mice received intraperitoneal injection of antibodies (biotinylated, 20 mg/kg) in 0.1 mL DPBS. Blood was collected 24 hours after injection via tail vein and mice then received transcardial perfusion at 2 mL/min by DPBS for 10 min. The brain tissues were processed as described above for immunofluorescent staining or biochemical analysis.

### Measurement of Antibody Concentration in Brain and Serum.

High-binding ELISA plates (Corning) were coated with mouse TREM2 (Sino Biological) at 2 μg/mL overnight at 4° C. After blocking with 1% BSA PBS, individual brain lysates were incubated with coated TREM2 for 2 hours at room temperature. After washing with PBS+0.05% Tween-20, anti-mouse Fc-HRP (Jackson Immunoresearch) was added at 1:5000 concentration and incubated for 1 hour at room temperature. After washing with PBS+0.05% Tween-20, TMB substrate (Termo Fisher Scientific) was added and incubated for 5 min before being stopped by 1N H₂SO₄. OD values were read at 450 nm. Standard curves were established using purified corresponding bispecific antibodies following the same method as described above.

TABLE 1
Heavy and light chain pairs used for TREM2 mAb expression.

| mAb Name | Heavy chain | light chain |
|---|---|---|
| TREM2-Ab1Rb | 1 H | 1 k |
| TREM2-Ab2Rb | 2 H | 2 k |
| TREM2-Ab6Rb | 2 H | 6 k |
| TREM2- | 12 H | 12 k |
| Ab12Rb | | |
| TREM2- | 16 H | 16 k |
| Ab16Rb | | |
| TREM2- | 22 H | 22 k |
| Ab22Rb | | |
| TREM2- | 26 H | 26 k |
| Ab26Rb | | |
| TREM2-Ab2Hu | H2-Hu | 2L-Hu |
| TREM2-Ab8Hu | H8-Hu | 8L-Hu |
| TREM2- | H19-Hu | 19L-Hu |
| Ab19Hu | | |

TABLE 2
Kinetic binding parameters of TREM2 mAbs determined using
biolayer interferometry (BLI) assay with Octet Red96.

| Antibody Name | K_{D} (M) | kₒₙ(1/Ms) | k_{off}(1/s) | R² |
|---|---|---|---|---|
| TREM2-Ab2Hu | 1.81E−08 | 3.59E+05 | 6.47E−03 | 0.9563 |
| TREM2-Ab8Hu | 8.18E−09 | 6.09E+05 | 4.99E−03 | 0.9274 |
| TREM2-Ab19Hu | 1.89E−08 | 5.22E+05 | 9.86E−03 | 0.9710 |
| TREM2-Ab1Rb | 1.42E−09 | 5.13E+05 | 7.27E−04 | 0.9888 |
| TREM2-Ab2Rb | 1.20E−09 | 3.76E+05 | 4.51E−04 | 0.9896 |
| TREM2-Ab6Rb | 1.97E−09 | 4.61E+05 | 9.09E−04 | 0.9784 |
| TREM2-Ab12Rb | 7.18E−08 | 9.55E+04 | 6.85E−03 | 0.9806 |
| TREM2-Ab16Rb | 3.20E−10 | 2.45E+05 | 7.84E−05 | 0.9905 |
| TREM2-Ab22Rb | 3.10E−09 | 3.46E+05 | 1.07E−03 | 0.9868 |
| TREM2-Ab26Rb | 2.75E−08 | 1.77E+05 | 4.87E−03 | 0.9843 |

TABLE 3
Epitope Bins grouped based on competition
binding using BLI Octet assay

| Epitope Bins | Antibody grouped in each bins |
|---|---|
| 1 | TREM2-Ab2Hu |
| | TREM2-Ab19Hu |
| 2 | TREM2-Ab8Hu |
| 3 | TREM2-Ab12Rb |
| | TREM2-Ab26Rb |
| 4 | TREM2-Ab16Rb |

TABLE 4
Amino Acid Sequences of Antibody Heavy (H) Chain CDRs

| Sequence Label | | |
|---|---|---|
| (mAb Source) | Sequence ID | Amino Acid Sequence |
| 1H-HCDR1-AA | (SEQ ID NO: 1) | GIDFSNYG |
| (TREM2-Ab1Rb) | | |
| 1H-HCDR2-AA | (SEQ ID NO: 2) | IYPDYGAT |
| (TREM2-Ab1Rb) | | |
| 1H-HCDR3-AA | (SEQ ID NO: 3) | ARSTGWGDFNL |
| (TREM2-Ab1Rb) | | |
| 2H-HCDR1-AA | (SEQ ID NO: 4) | GIDFSSAG |
| (TREM2-Ab2Rb) | | |
| 2H-HCDR2-AA | (SEQ ID NO: 5) | IYPDYGVT |
| (TREM2-Ab2Rb) | | |
| 2H-HCDR3-AA | (SEQ ID NO: 6) | ARMDAADAGYGYDL |
| (TREM2-Ab2Rb) | | |
| 12H-HCDR1-AA | (SEQ ID NO: 7) | GFSFSSYYF |
| (TREM2-Ab12Rb) | | |
| 12H-HCDR2-AA | (SEQ ID NO: 8) | IYAGTSANT |
| (TREM2-Ab12Rb) | | |
| 12H-HCDR3-AA | (SEQ ID NO: 9) | ARDRGYYTYADAGYAYATDGFDL |
| (TREM2-Ab12Rb) | | |
| 16H-HCDR1-AA | (SEQ ID NO: 10) | GFSFSDTAY |
| (TREM2-Ab16Rb) | | |
| 16H-HCDR2-AA | (SEQ ID NO: 11) | IAFGDGR |
| (TREM2-Ab16Rb) | | |
| 16H-HCDR3-AA | (SEQ ID NO: 12) | ASNFTL |
| (TREM2-Ab16Rb) | | |
| 22H-HCDR1-AA | (SEQ ID NO: 13) | GFSFSSTA |
| (TREM2-Ab22Rb) | | |
| 22H-HCDR2-AA | (SEQ ID NO: 14) | IGFSSGI |
| (TREM2-Ab22Rb) | | |
| 22H-HCDR3-AA | (SEQ ID NO: 15) | ARFFNL |
| (TREM2-Ab22Rb) | | |
| 26H-HCDR1-AA | (SEQ ID NO: 16) | GFSFSYSYY |
| (TREM2-Ab26Rb) | | |
| 26H-HCDR2-AA | (SEQ ID NO: 17) | IYGGGSGSS |
| (TREM2-Ab26Rb) | | |
| 26H-HCDR3-AA | (SEQ ID NO: 18) | ARIDDAYATVM |
| (TREM2-Ab26Rb) | | |
| H2-Hu-HCDR1-AA | (SEQ ID NO: 19) | GYTFTSYY |
| (TREM2-Ab2Hu) | | |
| H2-Hu-HCDR2-AA | (SEQ ID NO: 20) | INPSGGST |
| (TREM2-Ab2Hu) | | |
| H2-Hu-HCDR3-AA | (SEQ ID NO: 21) | ARDRPGRSSIAAAGPKRGHYGMDV |
| (TREM2-Ab2Hu) | | |
| H8-Hu-HCDR1-AA | (SEQ ID NO: 22) | GVSVSNNY |
| (TREM2-Ab8Hu) | | |
| H8-Hu-HCDR2-AA | (SEQ ID NO: 23) | MYSDATT |
| (TREM2-Ab8Hu) | | |
| H8-Hu-HCDR3-AA | (SEQ ID NO: 24) | ARESVAGTLDF |
| (TREM2-Ab8Hu) | | |
| H19-Hu-HCDR1- | (SEQ ID NO: 25) | GYTFTSYY |
| AA | | |
| (TREM2-Ab19Hu) | | |
| H19-Hu-HCDR2- | (SEQ ID NO: 26) | INPSGGST |
| AA | | |
| (TREM2-Ab19Hu) | | |
| H19-Hu-HCDR3- | (SEQ ID NO: 27) | ARSLSSGSYYDY |
| AA | | |
| (TREM2-Ab19Hu) | | |

TABLE 5
Amino Acid Sequences of Antibody Light (L) Chain CDRs

| Sequence Label | | |
|---|---|---|
| (mAb Source) | Sequence ID | Amino Acid Sequence |
| 1K-LCDR1-AA | (SEQ ID NO: 28) | QNIGSN |
| (TREM2-Ab1Rb) | | |
| 1K-LCDR3-AA | (SEQ ID NO: 29) | QCTYYGSSYVA |
| (TREM2-Ab1Rb) | | |
| 2K-LCDR1-AA | (SEQ ID NO: 30) | QSVGSN |
| (TREM2-Ab2Rb) | | |
| 2K-LCDR3-AA | (SEQ ID NO: 31) | HSYYYSSTSADA |
| (TREM2-Ab2Rb) | | |
| 6K-LCDR1-AA | (SEQ ID NO: 32) | QSVGTN |
| (TREM2-Ab6Rb) | | |
| 6K-LCDR3-AA | (SEQ ID NO: 33) | HSYYYSSTSADA |
| (TREM2-Ab6Rb) | | |
| 12K-LCDR1-AA | (SEQ ID NO: 34) | QNIYSN |
| (TREM2-Ab12Rb) | | |
| 12K-LCDR3-AA | (SEQ ID NO: 35) | QSNYYSSSSNYGPNA |
| (TREM2-Ab12Rb) | | |
| 16K-LCDR1-AA | (SEQ ID NO: 36) | QSIGNN |
| (TREM2-Ab16Rb) | | |
| 16K-LCDR3-AA | (SEQ ID NO: 37) | QCTYASSAT |
| (TREM2-Ab16Rb) | | |
| 22K-LCDR1-AA | (SEQ ID NO: 38) | ETVYSNDR |
| (TREM2-Ab22Rb) | | |
| 22K-LCDR3-AA | (SEQ ID NO: 39) | AGGYNGNIWA |
| (TREM2-Ab22Rb) | | |
| 26K-LCDR1-AA | (SEQ ID NO: 40) | EDIYSS |
| (TREM2-Ab26Rb) | | |
| 26K-LCDR3-AA | (SEQ ID NO: 41) | QNYYYVRGADYWA |
| (TREM2-Ab26Rb) | | |
| 2L-Hu-LCDR1-AA | (SEQ ID NO: 42) | SSDVGGYNY |
| (TREM2-Ab2Hu) | | |
| 2L-Hu-LCDR3-AA | (SEQ ID NO: 43) | SSYTSSRTLV |
| (TREM2-Ab2Hu) | | |
| 8L-Hu-LCDR1-AA | (SEQ ID NO: 44) | SSNIGTYP |
| (TREM2-Ab8Hu) | | |
| 8L-Hu-LCDR3-AA | (SEQ ID NO: 45) | ATWGDSLNAVV |
| (TREM2-Ab8Hu) | | |
| 19L-Hu-LCDR1-AA | (SEQ ID NO: 46) | SSDVGGYNY |
| (TREM2-Ab19Hu) | | |
| 19L-Hu-LCDR3-AA | (SEQ ID NO: 47) | SSYTSSRTLV |
| (TREM2-Ab19Hu) | | |

TABLE 6
Amino Acid Sequences of Antibody Heavy (H) Chains

| Sequence Label | | |
|---|---|---|
| (mAb Source) | Sequence ID | Amino Acid Sequence |
| H2-Hu-HC-AA | (SEQ ID NO: 48) | QVQLVQSGAEVKKPGASVKVSCKASGYTFTS |
| (TREM2-Ab2Hu) | | YYMHWVRQAPGQGLEWMGIINPSGGSTSYA |
| | | QKFQGRVTMTRDTSTSTVYMELSSLRSEDTA |
| | | VYYCARDRPGRSSIAAAGPKRGHYGMDVWG |
| | | QGTTVSVSS |
| H8-Hu-HC-AA | (SEQ ID NO: 49) | QVQLVESGGGLIQPGGSLRLSCAVSGVSVSN |
| (TREM2-Ab8Hu) | | NYMNWVRQAPGKGLEWVSSMYSDATTHYA |
| | | DSVKGRFTISRDNSKNNMYLQMDSLRAEDTA |
| | | VYYCARESVAGTLDFWGQGTTVSVSS |
| H19-Hu HC-AA | (SEQ ID NO: 50) | EVQLVQSGAEVKKPGASVKVSCKASGYTFTS |
| (TREM2- | | YYMHWVRQAPGQGLEWMGIINPSGGSTSYA |
| Ab19Hu) | | QKFQGRVTMTRDTSTSTVYMELSSLRSEDTA |
| | | VYYCARSLSSGSYYDYWGQGTLVSVSS |
| 1H-HC-AA | (SEQ ID NO: 51) | QEQLVESGGGLVTPGGTLTLTCTASGIDFSNY |
| (TREM2-Ab1Rb) | | GISWVRQAPGKGLEWIGYIYPDYGATAYAN |
| | | WVNGRFTVSLDNAQNTVTLNMTSLTAADTA |
| | | TYFCARSTGWGDFNLWGPGTLVTVSS |
| 2H-HC-AA | (SEQ ID NO: 52) | QKQLVESGGGLVKPEGSLTLTCKVSGIDFSSA |
| (TREM2-Ab2Rb) | | GISWVRQAPGKGLEWIAYIYPDYGVTDYAN |
| | | WVNGRFTISLDNAQNTVFLQMTSLTAADTAT |
| | | YFCARMDAADAGYGYDLWGPGTLVTVSS |
| 12H-HC-AA | (SEQ ID NO: 53) | QSVEESGGDLVKSGASLTLTCTASGFSFSSYY |
| (TREM2-Ab12Rb) | | FMCWVRQAPGRGLEWIACIYAGTSANTYYA |
| | | SWAKGRFTISKTSSTTVTLQMTSLTAADTATY |
| | | FCARDRGYYTYADAGYAYATDGFDLWGPGT |
| | | LVTVSS |
| 16H-HC-AA | (SEQ ID NO: 54) | QEQLVESGGGLVKPGASLTLTCTASGFSFSDT |
| (TREM2-Ab16Rb) | | AYICWVRQAPGKGLEWVACIAFGDGRTYLA |
| | | SWAKGRFTISKTSSTTVTLQMTSLPAADTATY |
| | | FCASNFTLWGPGTLVTVSS |
| 22H-HC-AA | (SEQ ID NO: 55) | QSVEESGGDLVKPGASLTLTCTASGFSFSSTA |
| (TREM2-Ab22Rb) | | MCWVRQAPGKGLEWIACIGFSSGIINYANSV |
| | | KGRFTISKTSSTTVSLQMNSLTAADTATYFCA |
| | | RFFNLWGPGTLVTVSS |
| 26H-HC-AA | (SEQ ID NO: 56) | QKQLVESGGDLVKPEGSLTLTCTASGFSFSYS |
| (TREM2-Ab26Rb) | | YYMCWVRQAPGKGLEWIGCIYGGGSGSSYY |
| | | ASWAKGRFTISKTSSTTVTLQVTSLTAADTAT |
| | | YFCARIDDAYATVMWGPGTLVTVSS |

TABLE 7
Amino Acid Sequences of Antibody Light (L) Chains

| Sequence Label | | |
|---|---|---|
| (mAb Source) | Sequence ID | Amino Acid Sequence |
| 1K-LC-AA | (SEQ ID NO: 57) | DVVMTQTPASVSEPVGGTVTIKCQASQNIGSNL |
| (TREM2-Ab1Rb) | | AWYQQKPGQPPNLLIYGASNLASGVSSRFKGSS |
| | | SGTEYTLTISDLECADAATYYCQCTYYGSSYVA |
| | | FGGGTEVVVK |
| 2K-LC-AA | (SEQ ID NO: 58) | AAVLTQTTSPVSGAVGGTVTIKCQASQSVGSNL |
| (TREM2-Ab2Rb) | | AWYQQKPGQPPKILIGGASKLASGVPSRFSGSGS |
| | | GTEFTLTISDLECADAGTYYCHSYYYSSTSADAF |
| | | GGGTKVVVE |
| 6K LC-AA | (SEQ ID NO: 59) | DVVMTQTTSPVSGAVGGTVTIKCQASQSVGTNL |
| (TREM2-Ab6Rb) | | AWYQQKPGQPPKILIGGASKLASGVPSRFSGSGS |
| | | GTEFTFTISDLECADAGTYYCHSYYYSSTSADAF |
| | | GGGTEVVVK |
| 12K-LC-AA | (SEQ ID NO: 60) | ALVMTQTPASVEAAVGGTVTIKCQASQNIYSNL |
| (TREM2-Ab12Rb) | | AWYQQKPGQPPKRLIYKASTLPSGVPSRFKGSG |
| | | SGTQFTLTISDLECADAATYYCQSNYYSSSSNYG |
| | | PNAFGGGTEVVVK |
| 16K-LC-AA | (SEQ ID NO: 61) | DVVMTQTPASVSEPVGGTVTINCQAIQSIGNNFV |
| (TREM2-Ab16Rb) | | WYQQKPGQPPKLLIYKASIRASGVSSRFRGSGSG |
| | | TDFTLTISDLECDDTGTYYCQCTYASSATFGGGT |
| | | EVVVK |
| 22K-LC-AA | (SEQ ID NO: 62) | DPVLTQTPSSVSAAVGGTVTISCQATETVYSND |
| (TREM2-Ab22Rb) | | RLAWYQQKPGQPPKLLIYRISTLESGVPSRFKGS |
| | | GSGTQFTLTISGVQCDDAATYYCAGGYNGNIW |
| | | AFGGGTEVVVK |
| 26K-LC-AA | (SEQ ID NO: 63) | DVVMTQTASPVSGAVGGTVTIKCQASEDIYSSL |
| (TREM2-Ab26Rb) | | AWYQQKPGQPPKLLIYQASKLASGVPPRFSGSG |
| | | SGTEYTLTISDLECADAATYYCQNYYYVRGAD |
| | | YWAFGGGTEVVVE |
| 2L-LC-AA | (SEQ ID NO: 64) | QSALNQPASVSGSPGQSITISCTGTSSDVGGYNY |
| (TREM2-Ab2Hu) | | VSWYQQHPGKAPKLMIYEVSNRPSGVSNRFSGS |
| | | KSGNTASLTISGLQAEDEADYYCSSYTSSRTLVF |
| | | GTGTKVTVL |
| 8L-LC-AA | (SEQ ID NO: 65) | SYELTQPPSASGTPGQRVTISCSGSSSNIGTYPVN |
| (TREM2-Ab8Hu) | | WYQKLPGTAPKLLIYTNNQRPSGVPDRFSGSKS |
| | | GTSASLTISGLQSEDEADYYCATWGDSLNAVVF |
| | | GGGTKVTVL |
| 19L-LC-AA | (SEQ ID NO: 66) | QSALTQPASVSGSPGQSITISCTGTSSDVGGYNY |
| (TREM2-Ab19Hu) | | VSWYQQHPGKAPKLMIYDVTKRPSGVSNRFSG |
| | | SKSGNTASLTVSGLQAEDEADYYCSSYTSSRTL |
| | | VFGGGTKVTVL |

TABLE 8
DNA Sequences Encoding Antibody Heavy (H) Chain CDR sequences

| Sequence Label | Sequence ID | DNA Sequence |
|---|---|---|
| 1H-HCDR1-DNA | (SEQ ID NO: 67) | ggaatcgacttcagtaactatggc |
| 1H-HCDR2-DNA | (SEQ ID NO: 68) | atttatcctgattatggtgccaca |
| 1H-HCDR3-DNA | (SEQ ID NO: 69) | gcaagaagtactggctggggtgactttaacttg |
| 2H-HCDR1-DNA | (SEQ ID NO: 70) | ggaatcgacttcagtagcgctggc |
| 2H-HCDR2-DNA | (SEQ ID NO: 71) | atttatcctgattatggtgtcaca |
| 2H-HCDR3-DNA | (SEQ ID NO: 72) | gcaagaatggatgctgctgatgctggttatggttatgacttg |
| 12H-HCDR1-DNA | (SEQ ID NO: 73) | ggattctccttcagtagctactacttc |
| 12H-HCDR2-DNA | (SEQ ID NO: 74) | atttatgctggtactagtgctaacact |
| 12H-HCDR3-DNA | (SEQ ID NO: 75) | gcgagagatcggggttactatacttatgctgatgctggttat |
| | | gcttatgcaactgatggttttgatctc |
| 16H-HCDR1-DNA | (SEQ ID NO: 76) | ggattctccttcagcgacaccgcgtac |
| 16H-HCDR2-DNA | (SEQ ID NO: 77) | attgcttttggtgatggcagg |
| 16H-HCDR3-DNA | (SEQ ID NO: 78) | gcgagtaattttacattg |
| 22H-HCDR1-DNA | (SEQ ID NO: 79) | ggattctccttcagtagcactgca |
| 22H-HCDR2-DNA | (SEQ ID NO: 80) | attggttttagtagtggtatt |
| 22H-HCDR3-DNA | (SEQ ID NO: 81) | gcgagattctttaatttg |
| 26H-HCDR1-DNA | (SEQ ID NO: 82) | ggattctctttcagttacagctactac |
| 26H-HCDR2-DNA | (SEQ ID NO: 83) | atttatggtggtggtagtggtagttct |
| 26H-HCDR3-DNA | (SEQ ID NO: 84) | gcgagaattgatgatgcttatgctactgtcatg |
| H2-Hu-HCDR1-DNA | (SEQ ID NO: 85) | ggatacaccttcaccagctactat |
| H2-Hu-HCDR2-DNA | (SEQ ID NO: 86) | atcaaccctagtggtggtagcaca |
| H2-Hu-HCDR3-DNA | (SEQ ID NO: 87) | gcgagagatcgaccgggccgttccagtatagcagcagct |
| | | gggccgaagcggggccactacggtatggacgtc |
| H8-Hu-HCDR1-DNA | (SEQ ID NO: 88) | ggggtcagcgtcagtaacaactac |
| H8-Hu-HCDR2-DNA | (SEQ ID NO: 89) | atgtatagtgatgccactaca |
| H8-Hu-HCDR3-DNA | (SEQ ID NO: 90) | gcgagggaatcagtggctggcacgcttgacttc |
| H19-Hu-HCDR1-DNA | (SEQ ID NO: 91) | ggatacaccttcaccagctactat |
| H19-Hu-HCDR2-DNA | (SEQ ID NO: 92) | atcaaccctagtggtggtagcaca |
| H19-Hu-HCDR3-DNA | (SEQ ID NO: 93) | gcgagatccctgagcagtgggagctactatgactac |

TABLE 9
DNA Sequences Encoding Antibody Light (L) Chain CDR sequences

| Sequence Label | Sequence ID | DNA Sequence |
|---|---|---|
| 1K-LCDR1-DNA | (SEQ ID NO: 94) | caggtgcagctggtgcag |
| 1K-LCDR3-DNA | (SEQ ID NO: 95 | caatgtacttattatggtagtagttatgttgct |
| 2K-LCDR1-DNA | (SEQ ID NO: 96) | cagagcgttggtagtaat |
| 2K-LCDR3-DNA | (SEQ ID NO: 97) | cacagttattattatagtagtactagtgctgatgct |
| 6K-LCDR1-DNA | (SEQ ID NO: 98) | cagagcgttggtactaat |
| 6K-LCDR3-DNA | (SEQ ID NO: 99) | cacagttattattatagtagtactagtgctgatgct |
| 12K-LCDR1-DNA | (SEQ ID NO: 100) | cagaacatttacagcaat |
| 12K-LCDR3-DNA | (SEQ ID NO: 101) | caaagcaattattatagtagtagtagtaattatggccctaatgct |
| 16K-LCDR1-DNA | (SEQ ID NO: 102) | cagagcattggtaataat |
| 16K-LCDR3-DNA | (SEQ ID NO: 103) | caatgtacttatgctagtagtgctact |
| 22K-LCDR1-DNA | (SEQ ID NO: 104) | gagactgtttatagtaacgaccgc |
| 22K-LCDR3-DNA | (SEQ ID NO: 105) | gcaggcggttataatggtaatatttgggct |
| 26K-LCDR1-DNA | (SEQ ID NO: 106) | gaggacatttacagctct |
| 26K-LCDR3-DNA | (SEQ ID NO: 107) | cagaactattattatgttcgtggtgctgattattgggct |
| 2L-Hu-LCDR1-DNA | (SEQ ID NO: 108) | Agcagtgacgttggtggttataactat |
| 2L-Hu-LCDR3-DNA | (SEQ ID NO: 109) | agctcatatacaagcagccgcactctcgtc |
| 8L-Hu-LCDR1-DNA | (SEQ ID NO: 110) | agctccaacatcggaacttatcct |
| 8L-Hu-LCDR3-DNA | (SEQ ID NO: 111) | gcaacatggggtgacagcctgaatgctgtggta |
| 19L-Hu-LCDR1-DNA | (SEQ ID NO: 112) | agcagtgacgttggtggttataactat |
| 19L-Hu-LCDR3-DNA | (SEQ ID NO: 113) | agctcatatacaagcagtagaactttagtc |

### NFAT-GFP Reporter Assay.

The TREM2-DAP12 (DNAX-activation protein 12) reporter construct was generated by fusing mouse TREM2 (aa 19-171) with huDAP12 (aa 28-113) with D50A mutation. The original signal peptide of TREM2 was replaced by leader sequence from mouse immunoglobulin a light chain. A HA tag was introduced to the N-terminus of TREM2.

The reporter gene was cloned into pCDH-CMV-MCS-IRES-Puro. The 2B4 reporter cells transduced with individual reporter constructs were generated by lentivirus transduction. To prepare lentivirus particles, pCMV-VSV-G (Addgene 8454), pCMV delta R8.2 (Addgene 12263), and individual pCDH transfer plasmids containing GOI were transfected into HEK293T. The 2B4 NFAT-GFP parental reporter cells were transduced with lentivirus supernatant (1:1 diluted in RPMI-1640) overnight under the presence of 10 μg/mL polybrene (Santa Cruz Biotechnology). After 48 hours of transduction, cells were selected with 1 μg/mL puromycin until a sufficient number of cells with transgene emerged.

For the reporter assay, ligands were coated onto 96-well cell culture plates at their optimal concentrations determined in preliminary experiments: oAβ (1 μM in DPBS, overnight, 4° C.), PS (0.1 mg/mL in methanol, room temperature until fully evaporated), and PC (L-α-phosphatidylcholine, purchased from Avanti Polar Lipids, 0.03 mg/mL in methanol, room temperature until fully evaporated). After ligand coating, unbound ligands were removed by washing with DPBS 3 times. A total of 100,000 reporter cells were seeded into individual wells (96-well plate) in 0.1 mL complete medium with 1 μg/mL puromycin with designated soluble antibody treatments. After overnight culturing, GFP positive populations were read using an iQue3 high throughput flow cytometer (Sartorius) with at least 10,000 live cells collected.

Preparation of oAβ-Lipoprotein Complexes.

L-α-phosphatidylserine (PS) and 1,2-dimyristoyl-sn-glycero-3-phosphocholine (DMPC) were purchased from Avanti Polar Lipids as powder. PS and DMPC were dissolved in chloroform at 10 mg/mL and mixed at 1:4. Chloroform was evaporated under vacuum and formed a thin layer containing the mixture of PS and DMPC. DPBS was added to re-hydrate the lipid mixture to 5 mg/mL, and the liposomes were formed by sonication on ice until the solution becomes translucent. To prepare oAβ-lipoprotein complexes, PS/DMPC liposomes and apolipoprotein e (APOE) were mixed at final concentrations of 1 mg/mL for PS/DMPC liposomes and 0.25 mg/mL for APOE. The mixture was incubated at 18° C. 15 min and 30° C. 15 min for 3 cycles (Hubin, E., et al., Apolipoprotein E associated with reconstituted high-density lipoprotein-like particles is protected from aggregation. FEBS Lett, 2019. 593(11): p. 1144-1153). FAM-labeled oAβ was then added into the lapidated APOE at a final concentration of 1 μM and incubated at room temperature for 1 hr.

### Microglia Phagocytosis Assay.

Mouse neonatal microglia were prepared as previously described (Xiang, X., et al., TREM2 deficiency reduces the efficacy of immunotherapeutic amyloid clearance. EMBO Mol Med, 2016.8(9): p. 992-1004). For phagocytosis experiments, microglia were seeded in a poly-D-lysine coated 96-well plate in RMPI-1640 without serum or cytokines. oAβ-lipoprotein complex was diluted to a concentration equivalent to 100 nM FAM-oAβ with 1% BSA. The medium in the cell culture plate was replaced with the diluted oAβ-lipoprotein complex and incubated at 37° C. for 2 hours. After phagocytosis, cells were detached by trypsin for 5 min, and cell surface-bound FAM-oAβ was quenched by adding trypan blue to 0.2% and incubated for 5 min. Cells were then transferred into a V-bottom 96-well plate and washed twice by 350 g 5 min centrifugation. For groups with cytochalasin d (CytoD) treatment, 10 μM CytoD was pre-incubated with cells for 30 min at 37° C. and constantly present during the phagocytosis experiment. The phagocytosis was quantified using an iQue3 high throughput flow cytometer (Sartorius).

### Microglia Migration in Transwell Assays.

Mouse neonatal microglia were prepared as previously described (Xiang, X., et al., TREM2 deficiency reduces the efficacy of immunotherapeutic amyloid clearance. EMBO Mol Med, 2016. 8(9): p. 992-1004). Cells were seeded in a transwell insert (PET membrane, 8 μm pore size, Corning 3374) in RMPI-1640 without serum or cytokines. Corresponding treatments were added into both the migration and receiver chambers at designated concentrations. Only the receiver (bottom) chambers contain a 0.5 μM oAβ-lipid complex. Cells were cultured for 24 hours at 37° C. with 5% CO₂. After incubation, cells were washed three times with DPBS, fixed in 4% PFA for 10 min, and then stained with 0.05% crystal violet for 10 min. Unbound crystal violet was removed by washing with DPBS, and the plate was allowed to air-dry. Cell number was quantified by eluting cell-bound crystal violet in 33% acetic acid in H₂O (100 rpm shaking, 10 min) according to the manufacturer's protocol and literature (Moore, C. S., et al., P2Y12 expression and function in alternatively activated human microglia. Neurol Neuroimmunol Neuroinflamm, 2015.2(2): p. e80). The amount of crystal violet was quantified by measuring absorbance at 590 nm using a plate reader. For quantifying migrated cells, unmigrated cells that remain inside the Transwell insert were removed using moistened cotton swabs. Migration percentage was calculated by dividing OD values of migrated cells over OD values of total cells. For imaging microglia migration, the assay was conducted similarly as mentioned above, except the microglia cells were pre-labeled with 1 μM CFSE (Thermo) for 15 min at 37° C. The migrated cells were imaged using Nikon Eclipse TE2000E Widefield Fluorescence Microscope.

### 5×FAD Animal Study.

The animal experiments were conducted according to the institutional guidelines with approved protocols. 5×FAD mice (B6.Cg-Tg(APPSwFlLon,PSEN1*M146L*L286V)6799Vas/Mmjax, female, 8-week-old, MMRRC) were randomly grouped into 5 mice per group. After reaching 5-mo-old age, mice received 14 weekly intraperitoneal injections of antibodies in 0.2 mL DPBS. Two days after the last injection, mice were sacrificed and the brains were collected as described above for both immunofluorescence staining and biochemical analysis.

### Statistical Analysis.

GraphPad Prism (v8, GraphPad Software) was used to generate plots and perform statistical analysis. Statistical differences were determined to be significant at p<0.05 using a two-tailed Student t-test. Data are presented as mean f SD.

### Results

Ab18 Activates TREM2 without Interfering Ligand-TREM2 Interactions.

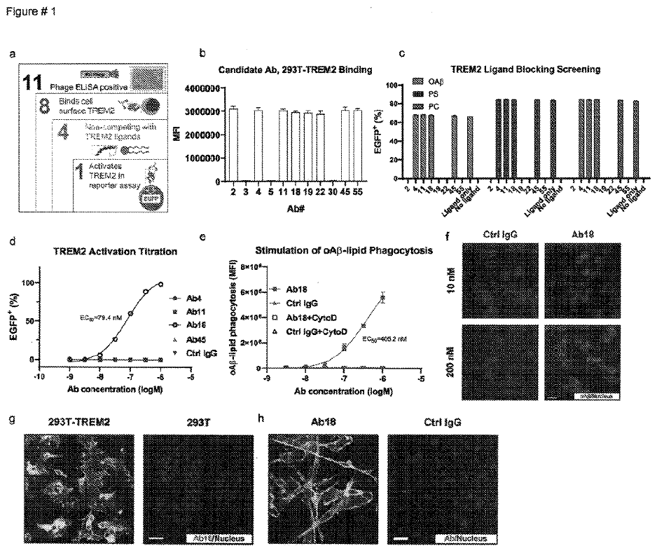

To generate agonist antibodies, the inventors designed an antibody screening scheme starting from panning a phage displayed human scFv library against the mouse TREM2 extracellular domain (ECD) (FIG. 1a). A phage ELISA assay was used to identify clones that can bind to TREM2 ECD; and 11 positive scFv clones were converted into human IgG1 antibodies for further studies and validation. The inventors first tested the 11 IgG1 antibodies for binding to cell surface-expressed TREM2 using flow cytometry. At least eight of the 11 IgG1 antibodies (e.g., Ab 2, Ab 4, Ab 11, Ab 18, Ab 19, Ab 22, Ab 45, and Ab 55) showed strong TREM2-dependent binding to HEK293T cells expressing TREM2 (FIG. 1b).

Natural ligands of TREM2 include oAβ and phospholipids (e.g., PC, PS). Interactions between TREM2 and those ligands have been shown to modulate microglia functions such as clustering around plaques, microglia metabolism and survival, and plaque-associated microgliosis (Wang, Y., et al., TREM2 lipid sensing sustains the microglial response in an Alzheimer's disease model. Cell, 2015. 160(6): p. 1061-71). Therefore, tests were done to identify agonist antibodies that do not compete with natural ligands in order to avoid perturbing the normal TREM2 signaling. An NFAT-EGFP reporter cell system similar to those previously described (Wang, Y., et al., TREM2 lipid sensing sustains the microglial response in an Alzheimer's disease model. Cell, 2015. 160(6): p. 1061-71; Song, W., et al., Alzheimer's disease-associated TREM2 variants exhibit either decreased or increased ligand-dependent activation. Alzheimers Dement, 2017. 13(4): p. 381-387) was constructed to express TREM2-DAP12. Ligand-TREM2 binding triggers the TREM2 signaling through the immunoreceptor tyrosine-based activation motif (ITAM) regions of DAP12, which further activates SYK and then induces a series of signaling cascades eventually leading to the expression of EGFP downstream of NFAT-responsible elements (Ohtsuka, M., et al., NFAM1, an immunoreceptor tyrosine-based activation motif-bearing molecule that regulates B cell development and signaling. Proc Natl Acad Sci USA, 2004. 101(21): p. 8126-31). Using the reporter assay, the eight antibodies that bind to TREM2 expressed on HEK293T cells were screened for their antagonism against the three representative TREM2 ligands, oAβ, PC, and PS, AM, Ab11, Ab18, and Ab45 showed no antagonism against any of the three ligands in the TREM2-DAP12 reporter assay (FIG. 1c). Four antibodies were then screened by flow cytometry for their agonist activity in the absence of TREM2 ligands in the TREM2-DAP12 NFAT EGFP reporter cell assay. Among the four antibodies, Ab18 showed concentration-dependent activation of TREM2 signaling with an EC₅₀ of 79.4 nM (FIG. 1d). Notably, the TREM2 activation activity of Ab18 require no solid surface coating or involvement of the Fc receptors, as the antibody was added as a soluble molecule in the culture medium and the Fc region bears the LALAPG mutations to abolish interactions with Fc receptors (Wang, X., M. Mathieu, and R. J. Brezski, IgG Fc engineering to modulate antibody effector functions. Protein & cell, 2018. 9(1): p. 63-73).

TREM2 signaling has been shown to promote microglial phagocytosis of amyloid β-lipid complexes (Yeh, F. L., et al., TREM2 Binds to Apolipoproteins. Including APOE and CLU/APOJ, and Thereby Facilitates Uptake of Amyloid-Beta by Microglia. Neuron, 2016. 91(2): p. 328-40). Ab18 was then tested to see whether it could enhance the phagocytosis of oAβ-lipid complexes by microglia. Mouse neonatal microglia, which are commonly used in microglia in vitro functional assays (Svoboda, D. S., et al., Human iPSC-derived microglia assume a primary microglia-like state after transplantation into the neonatal mouse brain. Proceedings of the National Academy of Sciences, 2019. 116(50): p. 25293), were used. Ab18-treated microglia showed a concentration-dependent increase of oAβ-lipid phagocytosis with an EC₅₀ of 405.2 nM, and treatment with the phagocytosis inhibitor CytoD was included as a control (FIG. 1e). The enhanced phagocytosis was further validated by immunofluorescence with fluorescent-labeled oAβ-lipid. Microglia treated by Ab18 at 200 nM showed a significant increase of oAβ-lipid phagocytosis over Ctrl IgG-treated microglia (FIG. 1f). However, Ab18 at 10 nM showed no effect in promoting oAβ-lipid phagocytosis as compared to Ctrl IgG (FIG. 1f). Using immunofluorescent staining, Ab18 showed strong cell surface binding to TREM2-expressing HEK293T cells (FIG. 1g). Using immunofluorescent staining, Ab18 also demonstrated strong cell surface binding of mouse neonatal microglia (FIG. 1h).

### Engineering Ab18 to Tetravalent Improves TREM2 Activation by 100-Fold.

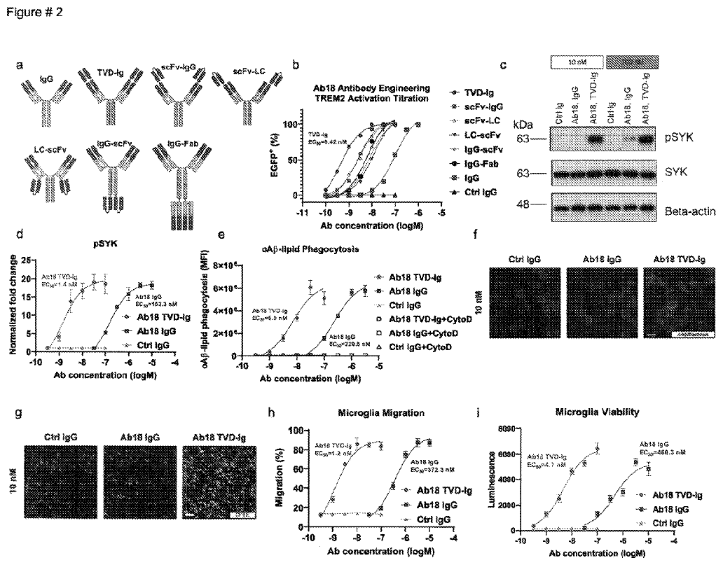

Although Ab18 demonstrates TREM2 agonist activity, the EC₅₀ is too high to reach in the brain under the conditions used in the assay due to poor antibody penetration through the blood-brain barrier (BBB). It has been reported that less than 0.1% of the antibody administered peripherally can enter the CNS and reaches a concentration of about 1 nM (Banks, W. A., From blood-brain barrier to blood-brain interface: new opportunities for CNS drug delivery. Nat Rev Drug Discov, 2016. 15(4): p. 275-92). Antibody engineering was used to increase the TREM2 agonist potency of Ab18. It was reported that increasing valency from bivalent to tetravalent using a format such as IgG-scFv may improve the crosslinking of receptors and thus the potency (Yang, Y., et al., Tetravalent biepitopic targeting enables intrinsic antibody agonism of tumor necrosis factor receptor superfamily members. MAbs, 2019. 11(6): p. 996-1011). Five different formats of Ab18 were engineered with tetravalency (FIG. 2a) to determine whether increased valency could increase the potency of the engineered antibodies. All of the engineered antibodies have tetravalency binding against TREM2. The engineered antibodies were titrated for activating TREM2 signaling using the TREM2-DAP12 reporter system. All five formats showed significantly improved TREM2 activation over the original Ab18; and the fold-of-increase ranged from 10 to 100-fold for the tetra variable domain immunoglobulin (TVD-Ig) format with an EC₅₀=0.42 nM (FIG. 2b).

TREM2 signaling triggers the phosphorylation of SYK (Ulland, T. K. and M. Colonna, TREM2—a key player in microglial biology and Alzheimer disease. Nat Rev Neurol, 2018. 14(11): p. 667-675). The effect of Ab18 TVD-Ig TREM2 signaling in microglia was tested by quantifying pSYK level change. Ab18 TVD-Ig-treated microglia showed a significant increase of phosphorylated SYK at both 10 nM and 100 nM concentrations (FIG. 2c). In contrast, microglia treated by the original Ab18 in IgG format showed a minimal increase of pSYK level at the 100 nM concentration but not at the 10 nM concentration in comparison to Ctrl Ig (FIG. 2c). Further titration showed a concentration-dependent increase of pSYK levels by both Ab18 TVD-Ig (EC₅₀=1.4 nM) and Ab18 IgG (EC₅₀=152.3 nM) in comparison to Ctrl IgG treatment. Notably, Ab18 TVD-Ig showed a 109-fold increase in activating TREM2 signaling over the original Ab18 (FIG. 2d).

The effect of Ab18 TVD-Ig on oAβ-lipid microglial phagocytosis was tested. Ab18 TVD-Ig-treated microglia showed a concentration-dependent increase of oAβ-lipid phagocytosis; and the EC₅₀ value of 6.9 for Ab18 TVD-Ig represents a 33-fold increase in improving oAβ-lipid phagocytosis over the original Ab18 IgG (FIG. 2e). Immunofluorescence imaging showed that Ab18 IgG at 10 nM had no improvement of oA-lipid microglial phagocytosis in comparison to Ctrl IgG, in contract, Ab18 TVD-Ig at 10 nM exhibited a significant increase in oAβ-lipid microglial phagocytosis (FIG. 2f).

In addition to phagocytosis, TREM2 is critical in regulating microglia migration toward amyloid. The migration of microglia toward amyloid is a key step in the microglia-mediated attenuation of plaque toxicity and Aβ removal (Wang, Y., et al., TREM2lipid sensing sustains the microglial response in an Alzheimer's disease model. Cell, 2015. 160(6): p. 1061-71). Microglia migration is also frequently used as a marker to assess microglia functions (Zhong, L., et al., Soluble TREM2 ameliorates pathological phenotypes by modulating microglial functions in an Alzheimer's disease model. Nature Communications, 2019. 10(1): p. 1365; Abud, E. M., et al., iPSC-Derived Human Microglia-like Cells to Study Neurological Diseases. Neuron, 2017. 94(2): p. 278-293.e9). At 10 nM concentration, Ab18 TVD-Ig significantly improved the migration of microglia toward oAβ-lipid; in contrast, effect of the original Ab18 IgG at the same concentration is similar to the negative control (FIG. 1g). Further titrations showed an EC₅₀ value of 1.2 nM for Ab18 TVD-Ig in contrast to the EC₅₀ value of 372.3 nM for the original Ab18 IgG (FIG. 2h).

TREM2 signaling promotes microglia survival under CSF depletion conditions (Wang, Y., et al., TREM2 lipid sensing sustains the microglial response in an Alzheimer's disease model. Cell, 2015. 160(6): p. 1061-71), and the synergy between TREM2 and CSF1R plays a role in plaque-associated microgliosis (Wang, Y., et al., TREM2 lipid sensing sustains the microglial response in an Alzheimer's disease model. Cell, 2015. 160(6): p. 1061-71). The inventors studied whether Ab18 TVD-Ig can improve microglia survival under low CSF supplementation (5 ng/mL) for 5 days. As shown in FIG. 2i, Ab18 TVD-Ig showed significantly stronger promotion of microglia survival as determined by an ultra-sensitive luminescence assay measuring ATP levels in live cells, and the EC₅₀ values were 4.7 nM and 466.3 nM for Ab18 TVD-Ig and the original Ab18 IgG, respectively, which represents a 99-fold improvement.

Tetravalent TREM2 Binding Effectively Triggers TREM2 Clustering without Altering Cellular TREM2 Levels.

TREM2 is associated with DAP12 that bears ITAM. Activation of the TREM2 leads to the phosphorylation in DAP12 ITAM regions and recruitment of SYK, which leads to the initiation of a number of signaling cascades (Ulrich, J. D., et al., Elucidating the Role of TREM2 in Alzheimer's Disease. Neuron, 2017. 94(2): p. 237-248). Efficient initiation of ITAM-mediated signaling activation often includes the clustering of receptors by multimeric ligands (Blank, U., et al., Inhibitory ITAMs as novel regulators of immunity. Immunol Rev, 2009. 232(1): p. 59-71). For example, TREM2 activation often includes ligand being coated on a solid surface or presented as a large multimeric complex (such as liposomes) (Schlepckow, K., et al., Enhancing protective microglial activities with a dual function TREM2 antibody to the stalk region. EMBO Mol Med, 2020. 12(4): p. e11227; Song, W., et al., Alzheimer's disease-associated TREM2 variants exhibit either decreased or increased ligand-dependent activation. Alzheimers Dement, 2017. 13(4): p. 381-387; Yeh, F. L., et al., TREM2 Binds to Apolipoproteins. Including APOE and CLU/APOJ, and Thereby Facilitates Uptake of Amyloid-Beta by Microglia. Neuron, 2016. 91(2): p. 328-40). It was shown in this study that the bivalent Ab18 IgG needs a relatively high concentration (EC₅₀=152.3 nanomolar) to activate TREM2; and in contrast the tetravalent engineered TVD-Ab18 exhibited significantly higher potency in TREM2 activation ((EC₅₀=1.4 nanomolar). The tetravalency-mediated enhancement of TREM2 activation was tested to determine whether it was a result of increased receptor clustering by directly assessing the clustering of TREM2 by the engineered antibodies.

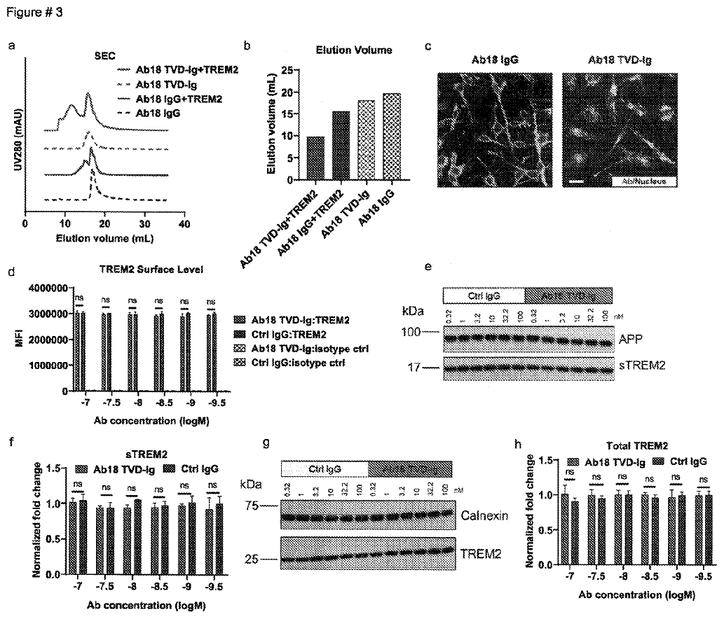

The clustering of TREM2 was studied by using size-exclusion chromatography to measure the molecular size of antibody-TREM2 complexes. The bivalent Ab18 IgG showed a clear complex formation between antibody and TREM2 with a retention time of about 15 min. In contrast, the tetravalent Ab18 TVD-Ig showed a significantly increased complex size as indicated by the reduced retention time to 10 min (FIGS. 3a and 3b), suggesting that the enhanced TREM2 activation by the tetravalent Ab18 TVD-Ig is associated with the increased clustering of TREM2. To further corroborate the SEC data, the clustering of TREM2 was studied in microglia using immunofluorescent imaging. Ab18 TVD-Ig treatment showed a significant number of dotted structures, which are likely the aggregated TREM2 molecules; and in contrast the dotted structures are largely absent in the bivalent Ab18-treated microglia (FIG. 3c). The combined results from the immunofluorescence imaging and the SEC analysis support the notion that tetravalent Ab18 TVD-Ig induces TREM2 clustering, which then triggers strong initiation of the ITAM-mediated signaling cascade.

It has been reported that TREM2 surface level is downregulated upon microglia activation due to protease cleavage and release of soluble TREM2 (sTREM2) fragments (Ulland, T. K. and M. Colonna, TREM2 —a key player in microglial biology and Alzheimer disease. Nat Rev Neurol, 2018. 14(11): p. 667-675). An antibody-based strategy was designed to enhance TREM2 signaling by blocking the α-secretase-mediated TREM2 shedding (Schlepckow, K., et al., Enhancing protective microglial activities with a dual function TREM2 antibody to the stalk region. EMBO Mol Med, 2020. 12(4): p. e11227). TREM2 level changes in microglia were quantified after Ab18 TVD-Ig treatment by multiple approaches. The change of cell surface TREM2 levels upon Ab18 TVD-Ig treatment was studied using flow cytometry. As shown in FIG. 3d, Ab18 TVD-Ig-treated microglia showed similar levels of TREM2 on the cell surface as compared to that of the Ctrl IgG and the Ab18 IgG. The same results were obtained for multiple antibody concentration points (FIG. 3d), which suggest that TREM2 activation by Ab18 TVD-Ig does not reduce cell surface TREM2 levels.

sTREM2 produced by TREM2 cleavage has been implicated as the biomarker of AD (Suárez-Calvet, M., et al., Early increase of CSF sTREM2 in Alzheimer's disease is associated with tau related-neurodegeneration but not with amyloid-β pathology. Molecular Neurodegeneration, 2019. 14(1): p. 1) and sTREM2 levels were found to correlate with plaque pathology (Zhong, L., et al., Soluble TREM2 ameliorates pathological phenotypes by modulating microglial functions in an Alzheimer's disease model. Nature Communications, 2019. 10(1): p. 1365; Vilalta, A., et al., Wild-type sTREM2 blocks A& #x3b2; aggregation and neurotoxicity, but the Alzheimer's R47H mutant increases A& #x3b2; aggregation. Journal of Biological Chemistry, 2021. 296). Increased sTREM2 production was observed with lipopolysaccharide (LPS) or interferon-gamma (IFNγ)-mediated myeloid cell activation (Ulland, T. K. and M. Colonna, TREM2 —a key player in microglial biology and Alzheimer disease. Nat Rev Neurol, 2018. 14(11): p. 667-675). In the present disclosure, sTREM2 levels in the supernatant of microglia culture were quantified after antibody treatment. Within the concentration range tested, Ab18 TVD-Ig-treated microglia showed similar levels of sTREM2 production as compared to that of the Ctrl IgG and the Ab18 IgG (FIGS. 3e and 3f), which suggest that TREM2 activation by Ab18 TVD-Ig does not increase sTREM2 production. In addition, the total TREM2 level in microglia treated with the engineered antibodies was determined. Within the concentration range tested, Ab18 TVD-Ig-treated microglia showed similar levels of total TREM2 as compared to that of the Ctrl IgG and the Ab18 IgG (FIGS. 3g and 3h), which indicate that TREM2 activation by Ab18 TVD-Ig does not change the total TREM2 level. Collectively, TREM2 activation by the tetravalent Ab18 TVD-Ig or bivalent Ab18 IgG showed similar effects on the cell surface TREM2, sTREM2, and total TREM2 levels.

### αTfR-Mediated Efficient TREM2 Antibody Brain Entry.

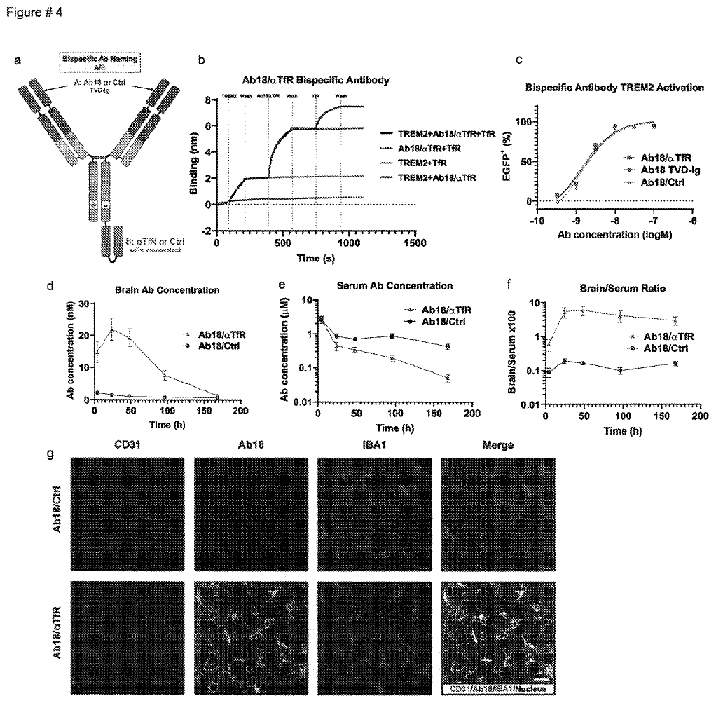

As demonstrated in FIG. 2b, the EC₅₀ of Ab18 TVD-Ig is in the 1-10 nM range in activating TREM2 signaling and promoting various microglia functions. However, the concentration of peripherally injected antibodies in brain parenchyma is usually lower than 1 nM (Banks, W. A., From blood-brain barrier to blood-brain interface: new opportunities for CNS drug delivery. Nat Rev Drug Discov, 2016. 15(4): p. 275-92). To increase the brain concentration of TREM2 antibodies, a bispecific antibody as constructed, the bispecific antibody consisting of the tetravalent TREM2 targeting Ab18 TVD-Ig and an antibody targeting the mouse transferrin receptor (αTfR) as a monovalent scFv in C-terminus of the one of the heavy chains (FIG. 4a). This bispecific antibody design takes advantage of the TfR transcytosis pathway to facilitate antibody delivery crossing the BBB (Banks, W. A., From blood-brain barrier to blood-brain interface: new opportunities for CNS drug delivery. Nat Rev Drug Discov, 2016. 15(4): p. 275-92; Pardridge, W. M., Drug Transport across the Blood-Brain Barrier. Journal of Cerebral Blood Flow & Metabolism, 2012. 32(11): p. 1959-1972; Kariolis, M. S., et al., Brain delivery of therapeutic proteins using an Fc fragment blood-brain barrier transport vehicle in mice and monkeys. Sci Transl Med, 2020. 12(545); Yu, Y. J., et al., Therapeutic bispecifc antibodies cross the blood-brain barrier in nonhuman primates. Sci Transl Med, 2014. 6(261): p. 261ra154; Yu, Y. J., et al., Boosting brain uptake of a therapeutic antibody by reducing its affinity for a transcytosis target. Sci Transl Med, 2011. 3(84): p. 84ra44; Niewoehner, J., et al., Increased brain penetration and potency of a therapeutic antibody using a monovalent molecular shuttle. Neuron, 2014.81(1): p. 49-60). It is noted that previous studies showed the mono-valency of αTfR is involved in effective brain entry, as bivalent antibodies targeting TfR were often trapped inside blood vasculature without entering the brain parenchyma (Niewoehner, J., et al., Increased brain penetration and potency of a therapeutic antibody using a monovalent molecular shuttle. Neuron, 2014. 81(1): p. 49-60).

### Monovalent αTfR Involves the Heterodimerization of Two Antibody-Heavy Chains (FIG. 4a).

Heterodimerization of antibody heavy chains was generated by the electrostatic steering strategy (Wang, F., et al., Design and characterization of mouse IgG1 and IgG2a bispecific antibodies for use in syngeneic models. MAbs, 2020. 12(1): p. 1685350). For long-term in vivo treatment, the immunogenicity of human IgG isotype involves either additional measures to dampen the immune system or using the mouse IgG isotype to avoid immunogenicity (Bohrmann, B., et al., Gantenerumab: a novel human anti-Aβ antibody demonstrates sustained cerebral amyloid-βbinding and elicits cell-mediated removal of human amyloid-β. J Alzheimers Dis, 2012. 28(1): p. 49-69). To avoid these complications in the present disclosure, the mouse IgG2a isotype with LALAPG mutations (L234A, L235A, and P329G) was used to abolish interactions with Fc receptors in our bispecific antibody design (Wang, X., M. Mathieu, and R. J. Brezski, IgG Fc engineering to modulate antibody effector functions. Protein & cell, 2018. 9(1): p. 63-73; Schlothauer, T., et al., Novel human IgG1 and IgG4 Fc-engineered antibodies with completely abolished immune effector functions. Protein Eng Des Sel, 2016. 29(10): p. 457-466). An electrostatic steering strategy, which allows the heterodimerization pairing of bispecific chains in mouse IgG isotypes (Wang, F., et al., Design and characterization of mouse IgG1 and IgG2a bispecific antibodies for use in syngeneic models. MAbs, 2020. 12(1): p. 1685350), was chosen for use in the present disclosure. The A/B format was used to name the various antibody designs, where A means the binding moieties at the N-terminus, and B means the binding moieties at the C-terminus. “A” could be Ab18 or Ctrl IgG in TVD-Ig format, and B could be the αTfR or Ctrl IgG in a monovalent scFv format (FIG. 4a). A special note is that although the TVD-Ig is not written, all the Ab18 used in the bispecific antibody studies are in the TVD-Ig format. To demonstrate the incorporation of TREM2 Ab and TfR Ab into a single molecule as in the bispecific constructs, the bispecific antibody was captured onto the sensor through first binding TREM2. After reaching equilibrium in blank buffer, the sensors with captured antibody were then dipped into a solution of TfR ECD. The results from this sandwich BLI assay validated the incorporation of both TREM2 Ab and TfR Ab into a single molecule (FIG. 4b). Three sets of “minus-one” controls that do not incorporate one of the three binding partners (TREM2, Ab, or TfR) were included in the sandwich BLI assay. As shown in curve Ab18/αTfR+TfR, omitting TREM2 showed a complete flat curve, which confirmed the observed binding signals are dependent on TREM2 (FIG. 4b) under the experimental conditions used. Similarly, in curve TREM2+TfR, omitting Ab showed a flat curve when the sensors were dipped into the TfR solution (FIG. 4b), which indicates the observed TfR binding signals were dependent on the antibody under the experimental conditions used. Finally, in curves TREM2+Ab18/αTfR, omitting TfR gave flat TfR binding, which confirmed the observed binding signals were from TfR. Taken together, all the Ab18/αTfR bispecific constructs contains both TfR and TREMe2 binding specificities.

To validate the bispecific antibody Ab18/αTfR maintains the ability to activate TREM2, the TREM2 NFAT-EGFP reporter cells were used to titrate TREM2 activation. As shown in FIG. 4c, the bispecific antibody Ab18/αTfR and Ab18/Ctrl showed similar activation of TREM2 reporter cells in comparison to Ab18 TVD-Ig, which indicates that the bispecific antibody engineering did not compromise functions of the TREM2 antibodies. Mice were injected with a single dose of the bispecific antibodies (Ab18/αTfR or Ab18/Ctrl) at 20 mg/kg intraperitoneally. The plasma and brain antibody concentrations were quantified at different time points by sandwich ELISA. To avoid contaminating antibodies from the blood, mice were perfused with DPBS before collecting the brains. As shown in FIG. 4d, starting from 4 hours, the Ab18/αTfR already demonstrated a 5-fold higher brain concentration than that of Ab18/Ctrl. The brain antibody concentration differences between Ab18/αTfR and Ab18/Ctrl continued to increase to 10-fold up to 24 hours post-injection. The concentration differences in the brain started to drop after 24 hours, and disappeared at day 7 post-injection. Over the time course, brain antibody concentration of Ab18/Ctrl was maintained at a low level of about 1 nM; and in contrast, the highest brain concentration of Ab18/αTfR reached more than 20 nM at 24 hours post-injection. In the serum, Ab18/αTfR showed a quicker clearance than that of Ab18/Ctrl, which could be attributed to the broad expression of TfR in the peripheral organs which mediated the faster clearance (FIG. 4e). Considering the quicker clearance of Ab18/αTfR and dependence of transcytosis in the serum [46, 47], the inventors calculated the brain/serum antibody concentration ratio and found an even more contrasting improvement of brain antibody entry by Ab18/αTfR with the highest increase of 30-fold at 24 hours post-injection (FIG. 4f). Even more notable is the brain/serum ratio of Ab18/αTfR continued to maintain above 10-fold over the level of Ab18/Ctrl even at day 7 post-injection (FIG. 4f).

As demonstrated previously by Niewoehner et al., TfR antibody may be trapped inside vasculature, and therefore the detected antibody in ELISA may be, at least partially, contributed by the antibody inside vasculature but not in the brain parenchyma. Immunofluorescence staining of floating brain slices from perfused mice was performed to validate the entry of TREM2 antibodies into the brain parenchyma. Ab18/αTfR treatment showed significant antibody distribution outside of the blood vessel as marked by CD31 staining, and in comparison, Ab18/Ctrl has almost no brain parenchyma staining possibly due to lower concentration (FIG. 4g). Moreover, immunostaining with microglia marker IBA1 showed the Ab18/αTfR signals co-localize with IBA1, indicating the TREM2 binding on microglia in the brain parenchyma (FIG. 4g). Taken together, these results demonstrate the efficient delivery of the Ab18/αTfR bispecific antibody crossing the BBB and significant TREM2 binding on microglia in the brain parenchyma.

### TREM2/αTfR Bispecific Antibody Reduces Plaque Burden in 5×FAD Mice.

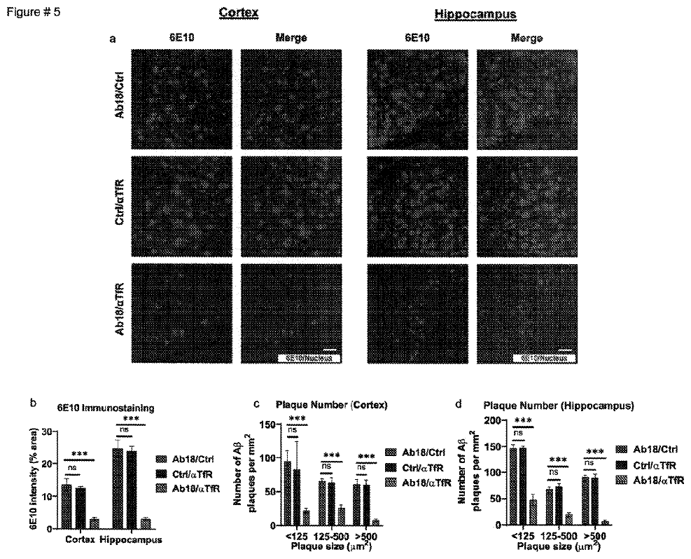

The present disclosure demonstrates that TREM2 agonism by Ab18 improves oAβ phagocytosis by microglia in vitro. In addition, long-term treatment of 5×FAD mice by Ab18/αTfR on amyloid pathology was studied. The study includes two control groups: Ab18/Ctrl (αTfR arm replaced by a control scFv that has no binding to TfR) and Ctrl/αTfR (the Ab18 TVD-Ig was replaced by a Ctrl IgG that does not bind TREM2, but the αTfR remains unchanged. The inventors started the antibody treatments when 5×FAD mice were 5 months old when the amyloid plaques already started to accumulate (Ghosh, A., et al., An epoxide hydrolase inhibitor reduces neuroinflammation in a mouse model of Alzheimer's disease. Sci Transl Med, 2020. 12(573); Forner, S., et al., Systematic phenotyping and characterization of the 5×FAD mouse model of Alzheimer's disease. Scientific Data, 2021. 8(1): p. 270). The 5×FAD mice were treated with the antibodies weekly by intraperitoneal injections at 20 mg/kg, which maintains an effective concentration of antibodies in the brain. After 14 weekly injections, the brains were collected after perfusion and the floating slices were stained by 6E10 to label Aβ plaques. Ab18/αTfR-treated mice showed significantly reduced overall plaque intensity, plaque number, and size in both cortex and hippocampus in comparison to that of Ab18/Ctrl and Ctrl/αTfR; and the two control groups (Ab18/Ctrl and Ctrl/αTfR) showed no significant differences from each other (FIG. 5a-d). In detail, further quantification showed about 4-fold and 7-fold decrease of the 6E10 immunostaining intensity upon treatment by Ab18/αTfR in the cortex and hippocampus, respectively. The overall plaque number in cortex showed 3-10-fold decrease in the cortex and hippocampus upon treatment by Ab18/αTfR. Treatment by Ab18/αTfR showed significant decrease of plaques with all sizes, with the decrease of large plaques over 500 μm² the most significant (about 10-fold in both the cortex and hippocampus). These results indicate that long-term treatment by Ab18/αTfR dramatically reduces both plaque number and size, and the efficacy is dependent on both Ab18 and αTfR.

TREM2 Antibody Promotes Microglia-Plaque Interactions without Affecting Astrocytes.

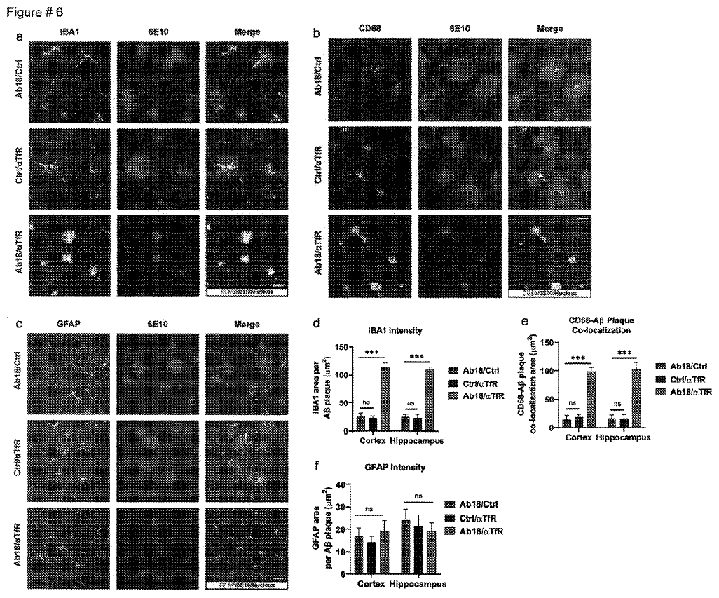

TREM2 has been shown to play key roles in microglia clusters around plaques and the subsequent removal of plaques (Wang, Y., et al., TREM2 lipid sensing sustains the microglial response in an Alzheimer's disease model. Cell, 2015. 160(6): p. 1061-71). In the present disclosure, the co-localization of microglia marker IBA1 with plaque marker 6E10 was studied to determine whether the Ab18/αTfR treatment improves the engagement of microglia with plaques. As shown in FIGS. 6a and 6d, IBA1 signals within 30 μm of plaques showed a significant 4-fold increase over that of Ab18/Ctrl or Ctrl/αTfR; which indicates that Ab18/αTfR significantly increased microglia clustering around plaques. This observation is consistent with in vitro results showing Ab18-mediated TREM2 activation promotes microglia migration toward the oAβ-lipid complex (FIG. 2g). After the clustering of microglia around plaques, the amyloid phagocytosis by microglia is the next crucial step in the removal of plaques. CD68, the phagocytic marker of microglia, is frequently used to study the phagocytic status of plaque proximal microglia (Wang, S., et al., Anti-human TREM2 induces microglia proliferation and reduces pathology in an Alzheimer's disease model. J Exp Med, 2020. 217(9); Ghosh, A., et al., An epoxide hydrolase inhibitor reduces neuroinflammation in a mouse model of Alzheimer's disease. Sci Transl Med, 2020. 12(573); Zhong, L., et al., Soluble TREM2 ameliorates pathological phenotypes by modulating microglial functions in an Alzheimer's disease model. Nature Communications, 2019. 10(1): p. 1365). CD68 and plaques (6E10) were stained and a significant increase (about 4-fold) of CD68 intensity around plaques in the Ab18/αTfR-treated mice over Ab18/Ctrl or Ctrl/αTfR (FIGS. 6b and 6e) was observed. This observation is consistent with in vitro results showing Ab18-mediated TREM2 activation promotes microglia phagocytosis of the oAβ-lipid complex. Astrocytes are known to play important roles in plaque pathology (Ghosh, A., et al., An epoxide hydrolase inhibitor reduces neuroinflammation in a mouse model of Alzheimer's disease. Sci Transl Med, 2020. 12(573); Forner, S., et al., Systematic phenotyping and characterization of the 5×FAD mouse model of Alzheimer's disease. Scientific Data, 2021. 8(1): p. 270; Gonzalez-Reyes, R. E., et al., Involvement of Astrocytes in Alzheimer's Disease from a Neuroinflammatory and Oxidative Stress Perspective. Frontiers in Molecular Neuroscience, 2017. 10(427)). To rule out the possibilities that TREM2 antibody affects astrocytes, GFAP (a marker of astrocytes) and 6E10 were co-stained. As shown in FIGS. 6c and 6f, GFAP-6E10 co-localization was similar across treatment groups, indicating that TREM2 agonism does not affect substantially astrocyte-plaque interactions. This result is consistent with the fact that expression of TREM2 is typically found microglia but not typically found in astrocytes.

### TREM2 Antibody Reduces Neuron Damages.

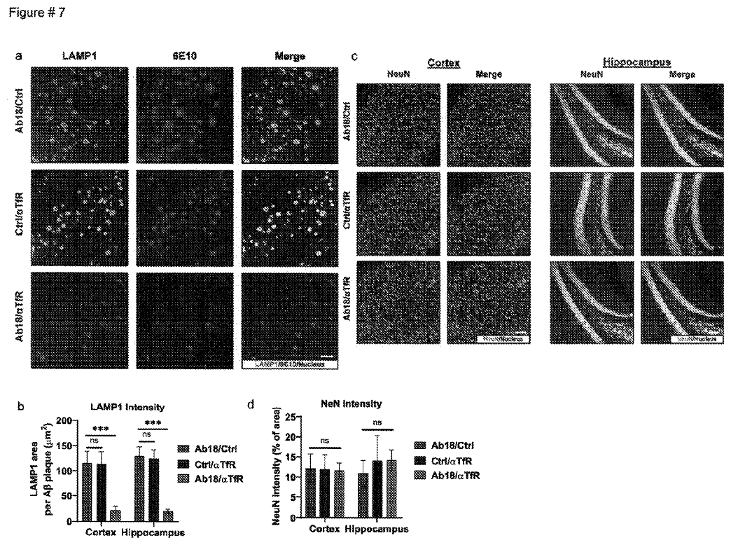

Neuron damage is severe in 5×FAD mice (Eimer, W. A. and R. Vassar, Neuron loss in the 5×FAD mouse model of Alzheimer's disease correlates with intraneuronal Aβ42 accumulation and Caspase-3 activation. Molecular Neurodegeneration, 2013. 8(1): p. 2). Plaques are known to associate with dystrophic neurites, which is a common pathologic feature of AD (Benzing, W. C., E. J. Mufson, and D. M. Armstrong, Alzheimer's disease-like dystrophic neurites characteristically associated with senile plaques are not found within other neurodegenerative disease unless amyloid β-protein deposition is present. Brain Research, 1993. 606(1): p. 10-18; Gowrishankar, S., et al., Massive accumulation of luminal protease-deficient axonal lysosomes at Alzheimer's disease amyloid plaques. Proc Natl Acad Sci USA, 2015.112(28): p. E3699-708; Sadleir, K. R., et al., Presynaptic dystrophic neurites surrounding amyloid plaques are sites of microtubule disruption. BACE1 elevation, and increased Aβ generation in Alzheimer's disease. Acta Neuropathol, 2016. 132(2): p. 235-256). The fact that plaques are surrounded by swollen, degenerating axons and dendrites (also known as dystrophic neurites) points the question whether the reduced plaque level by TREM2 agonism results in reduced dystrophic neurites. In the present disclosure, the cortex was stained with 6E10 and LAMP1, which is a marker dystrophic neurite (Zhong, L., et al., Soluble TREM2 ameliorates pathological phenotypes by modulating microglial functions in an Alzheimer's disease model. Nature Communications, 2019. 10(1): p. 1365; Forner, S., et al., Systematic phenotyping and characterization of the 5×FAD mouse model of Alzheimer's disease. Scientific Data, 2021. 8(1): p. 270; Gowrishankar, S., et al., Massive accumulation of luminal protease-deficient axonal lysosomes at Alzheimer's disease amyloid plaques. Proc Natl Acad Sci USA, 2015. 112(28): p. E3699-708). As shown in FIG. 7a-b, Ab18/αTfR treatment significantly decreased the intensity of LAMP1 surrounding the plaques and the overall number of LAMP1 clusters when compared with that of controls Ab18/Ctrl or Ctrl/αTfR. The effect of TREM2 agonism on the overall neuron density in the hippocampus and cortex was studied by immunostaining with NeuN, which is a neuronal nuclear antigen frequently used to quantify neuron density (Ghosh, A., et al., An epoxide hydrolase inhibitor reduces neuroinflammation in a mouse model of Alzheimer's disease. Sci Transl Med, 2020. 12(573); Mariani, M. M., et al., Neuronally-directed effects of RXR activation in a mouse model of Alzheimer's disease. Scientific Reports, 2017. 7(1): p. 42270) As shown in FIG. 7c-d, the overall intensity of NeuN in the hippocampus and cortex were similar between Ab18/αTfR, Ab18/Ctrl, and Ctrl/αTfR treatments, which suggest that TREM2 agonism significantly reduced the level of dystrophic neurites but did not alter the overall neuron density.

### Discussion

The Ab18 was first identified by screening for binding cell surface TREM2 and not blocking ligand-TREM2 interactions. TREM2-DAP12 reporter cell assay identified Ab18 as the candidate to show activation without the need to be coated on a solid surface or engage Fc receptors. In addition, the Ab18-treated microglia showed enhanced phagocytosis of oAβ-lipid. Observing the high EC₅₀ number, antibody format engineering was conducted and a tetravalent TVD-Ig format was identified with dramatically enhanced TREM2 activation. Multiple in vitro studies in microglia, including SYK activation, oAβ-lipid phagocytosis, migration toward oAβ-lipid, and microglia viability under CSF depletion, all showed the Ab18-TVD Ig has low nanomolar range effective concentrations. A series of mechanism studies showed the TVD-Ig format enhanced TREM2 activation through increased receptor clustering, but without affecting either cell surface or overall TREM2 levels in microglia. To further overcome the low antibody concentration brain entry, Ab18/αTfR was further engineered to be bispecific by taking advantage of the TfR-mediated transcytosis of macromolecules. Ab18/αTfR bispecific antibody demonstrated significantly enhanced antibody brain entry with proven in vivo microglia engagement. At last, the Ab18/αTfR bispecific antibody demonstrated superior activities in alleviating amyloid pathology in 5×FAD mice. Notably, the Ab18/αTfR bispecific antibody was able to reduce amyloid pathology in a treatment-related setting, with amyloid plaques already starting to develop. Immunofluorescence staining revealed enhanced microglia-plaque co-localization and plaque phagocytosis by microglia, which is likely to be the mechanism for reduced plaque pathology. Surprisingly, Ab18/αTfR also reduced the dystrophic neurite without affecting the overall neuron density. All the benefits show that TREM2 agonism by Ab18/αTfR is a viable approach in the treatment of AD and similar neurodegenerative diseases and disorders including, but, not limited to Alzheimer's Disease (AD), Parkinson's Disease (PD), dementia, dementia with Lewy bodies (DLB) and others, including neuroinflammatory processes and those involving microglia.

TABLE 10
DNA Sequences Encoding Variable Region of Antibody Heavy (H) chains

| Sequence Label | Sequence ID | DNA Sequence |
|---|---|---|
| H2-Hu-HC-DNA | (SEQ ID NO: 114) | caggtgcagctggtgcagtctggggctgaggtgaagaagcctgg |
| | | ggcctcagtgaaggtttcctgcaaggcatctggatacaccttcacc |
| | | agctactatatgcactgggtgcgacaggcccctggacaagggctt |
| | | gagtggatgggaataatcaaccctagtggtggtagcacaagctac |
| | | gcacagaagttccagggcagagtcaccatgaccagggacacgt |
| | | ccacgagcacagtctacatggagctgagcagcctgagatctgag |
| | | gacacggccgtgtattactgtgcgagagatcgaccgggccgttcc |
| | | agtatagcagcagctgggccgaagcggggccactacggtatgg |
| | | acgtctggggccaagggaccacggtcagcgtctcctca |
| H8-Hu-HC-DNA | (SEQ ID NO: 115) | caggtgcagctggtggagtctggaggaggcttgatccagcctgg |
| | | ggggtccctaagactctcctgtgccgtctctggggtcagcgtcagt |
| | | aacaactacatgaactgggtccgccaggctccagggaaggggct |
| | | ggagtgggtctcaagtatgtatagtgatgccactacacactatgca |
| | | gactccgtgaagggccgattcaccatctccagagacaattccaag |
| | | aacaatatgtatcttcaaatggacagcctgagagccgaagacacg |
| | | gccgtatattactgtgcgagggaatcagtggctggcacgcttgact |
| | | tctggggccaggggaccacggtcagcgtctcctca |
| 19H-Hu HC-DNA | (SEQ ID NO: 116) | gaggtgcagctggtgcagtctggggctgaggtgaagaagcctg |
| | | gggcctcagtgaaggtttcctgcaaggcatctggatacaccttcac |
| | | cagctactatatgcactgggtgcgacaggcccctggacaagggc |
| | | ttgagtggatgggaataatcaaccctagtggtggtagcacaagct |
| | | acgcacagaagttccagggtagagtcaccatgaccagggacac |
| | | gtccacgagcacagtctacatggagctgagcagcctgagatctg |
| | | aggacacggccgtgtattactgtgcgagatccctgagcagtggg |
| | | agctactatgactactggggccagggaaccctggtcagcgtctcc |
| | | tca |
| 16H-HC-DNA | (SEQ ID NO: 117) | caggagcagctggtggagtctggagggggtctggtcacgcctg |
| | | gaggaaccctgacactcacctgcacagcctctggaatcgacttca |
| | | gtaactatggcattagctgggtccgccaggctccagggaagggg |
| | | ctggagtggatcggatacatttatcctgattatggtgccacagccta |
| | | cgcgaactgggtgaatggccgattcaccgtctccctcgacaacgc |
| | | ccagaacacggtgactctgaatatgaccagtctgacagccgcgg |
| | | acacggccacctatttctgtgcaagaagtactggctggggtgactt |
| | | taacttgtggggcccaggcaccctggtcaccgtctcttca |
| 22H-HC-DNA | (SEQ ID NO: 118) | cagaagcagctggtggagtctggagggggtctggtcaagcctga |
| | | gggatccctgacactcacctgcaaagtctctggaatcgacttcagt |
| | | agcgctggcattagctgggtccgccaggctcccgggaaggggc |
| | | tggagtggatcgcatatatttatcctgattatggtgtcacagactacg |
| | | cgaactgggtgaatggccgattcaccatctccctcgacaacgccc |
| | | agaacacggtgtttctgcaaatgaccagtctgacagccgcggaca |
| | | ctgccacctatttctgtgcaagaatggatgctgctgatgctggttat |
| | | ggttatgacttgtggggcccaggcaccctggtcactgtctcctca |
| 26H-HC-DNA | (SEQ ID NO: 119) | cagtcggtggaggagtccgggggagacctggtcaagtctgggg |
| | | catccctgacactcacctgcacagcctctggattctccttcagtagc |
| | | tactacttcatgtgctgggtccgccaggctccagggaggggtctg |
| | | gagtggatcgcatgcatttatgctggtactagtgctaacacttacta |
| | | cgcgagctgggcgaaaggccgattcaccatctccaaaacctcgt |
| | | cgaccacggtgactctgcaaatgaccagtctgacagccgcggac |
| | | acggccacctatttctgtgcgagagatcggggttactatacttatgc |
| | | tgatgctggttatgcttatgcaactgatggttttgatctctggggccc |
| | | aggcaccctggtcaccgtctcttca |
| H2-Hu-HC-DNA | (SEQ ID NO: 120) | caggagcagctggtggagtccgggggaggcctggtcaagcctg |
| | | gggcatccctgacactcacctgcacagcttctggattctccttcag |
| | | cgacaccgcgtacatatgttgggtccgccaggctccagggaagg |
| | | ggctggagtgggtcgcatgcattgcttttggtgatggcaggacata |
| | | cttggcgagctgggcgaaaggccgattcaccatctccaaaacctc |
| | | gtcgaccacggtgactctgcaaatgaccagtctgccagccgcgg |
| | | acacggccacctatttctgtgcgagtaattttacattgtggggccca |
| | | ggcaccctggtcaccgtctcttca |
| H8-Hu-HC-DNA | (SEQ ID NO: 121) | cagtcggtggaggagtccgggggagacctggtcaagcctgggg |
| | | catccctgacactcacctgcacagcctctggattctccttcagtagc |
| | | actgcaatgtgctgggtccgccaggctccagggaaggggctgg |
| | | agtggatcgcttgcattggttttagtagtggtattattaactacgcga |
| | | actctgtgaagggccgattcaccatctccaagacctcgtcgacca |
| | | cggtgtctctgcaaatgaacagtctgacagccgcggacacggcc |
| | | acctatttctgtgcgagattctttaatttgtggggcccaggcaccctg |
| | | gtcaccgtctcttca |
| H19-Hu-HC-DNA | (SEQ ID NO: 122) | cagaagcagctggtggagtccgggggagacctggtcaagcctg |
| | | agggatccctgacactcacctgcacagcctctggattctctttcagt |
| | | tacagctactacatgtgctgggtccgccaggctccagggaaggg |
| | | gctggagtggatcggatgcatttatggtggtggtagtggtagttctt |
| | | actacgcgagctgggcgaaaggccgattcaccatctccaaaacc |
| | | tcgtcgaccacggtgactctgcaagtgaccagtctgacagccgc |
| | | ggacacggccacctatttctgtgcgagaattgatgatgcttatgcta |
| | | ctgtcatgtggggcccaggcaccctcgtcaccgtctcttca |

TABLE 11
DNA Sequences Encoding Variable Region of Antibody Light (L) chains

| Sequence Label | Sequence ID | DNA Sequence |
|---|---|---|
| 20L-LC-DNA | (SEQ ID NO: 123) | cagtctgccctgaatcagcctgcctccgtgtctgggtctcctggacagtcg |
| | | atcaccatctcctgcactggaaccagcagtgacgttggtggttataactatg |
| | | tctcctggtaccaacagcacccaggcaaagcccccaaactcatgatttatg |
| | | aggtcagtaatcggccctcaggggtttctaatcgcttctctggctccaagtct |
| | | ggcaacacggcctccctgaccatctctgggctccaggctgaggacgagg |
| | | ctgattattactgcagctcatatacaagcagccgcactctcgtcttcggaact |
| | | gggaccaaggtcaccgtccta |
| 8L-LC-DNA | (SEQ ID NO: 124) | tcctatgagctgactcagccaccctcagcgtctgggacccccgggcaga |
| | | gggtcaccatctcttgttctggaagtagctccaacatcggaacttatcctgtc |
| | | aactggtaccagaagctcccaggaacggcccccaagctcctcatctatac |
| | | taataatcagcggccctcaggggtccctgaccgattctctggctccaagtct |
| | | ggcacctcagcctccctgaccatcagtgggctccagtctgaggatgaggc |
| | | tgattattactgtgcaacatggggtgacagcctgaatgctgtggtattcggc |
| | | ggagggaccaaggtcaccgtccta |
| 19L LC-DNA | (SEQ ID NO: 125) | cagtctgccctgactcagcctgcctccgtgtctgggtctcctggacagtcg |
| | | atcaccatctcctgcactggaaccagcagtgacgttggtggttataactatg |
| | | tctcctggtaccaacagcacccaggcaaagcccccaaactcatgatttatg |
| | | atgtcactaagcggccctcaggggtttctaatcgcttctctggctccaagtct |
| | | ggcaacacggcctccctgaccgtctctgggctccaggctgaggacgagg |
| | | ctgattattactgcagctcatatacaagcagtagaactttagtcttcggcgga |
| | | gggaccaaggtcaccgtccta |
| 1K-LC-DNA | (SEQ ID NO: 126) | gatgtcgtgatgacccagactccagcctccgtgtctgaacctgtgggagg |
| | | cacagtcaccatcaagtgccaggccagtcagaatattggtagtaatttagc |
| | | ctggtatcagcagaaaccagggcagcctcccaacctcctgatctatggtg |
| | | catccaatctggcatctggggtctcatcgcggttcaaaggcagtagctctg |
| | | ggacagagtacactctcaccatcagcgacctggagtgtgccgatgctgcc |
| | | acttactactgtcaatgtacttattatggtagtagttatgttgctttcggcggag |
| | | ggaccgaggtggtcgtcaaa |
| 2K-LC-DNA | (SEQ ID NO: 127) | gcagccgtgctgacccagactacatcccccgtgtctggagctgtgggag |
| | | gcacagtcaccatcaagtgccaggccagtcagagcgttggtagtaatttag |
| | | cctggtatcagcagaaaccagggcagcctcccaagatcctgatcggtggt |
| | | gcatccaaactggcatctggggtcccatcgcggttcagtggcagtggatct |
| | | gggacagagttcactctcaccatcagcgacctggagtgtgccgatgctgg |
| | | cacttactactgtcacagttattattatagtagtactagtgctgatgctttcggc |
| | | ggagggaccaaggtggtcgtcgaa |
| 6K-LC-DNA | (SEQ ID NO: 128) | gatgtcgtgatgacccagactacatcccccgtgtctggagctgtgggagg |
| | | cacagtcaccatcaagtgccaggccagtcagagcgttggtactaatttagc |
| | | ctggtatcagcagaaaccagggcagcctcccaagatcctgatcggtggtg |
| | | catccaaactggcgtctggggtcccatcgcggttcagtggcagtggatctg |
| | | ggacagagttcactttcaccatcagcgacctggagtgtgccgatgctggc |
| | | acttactactgtcacagttattattatagtagtactagtgctgatgctttcggcg |
| | | gagggaccgaggtggtcgtcaaa |
| 12K-LC-DNA | (SEQ ID NO: 129) | gcccttgtgatgacccagactccagcctccgtggaggcagctgtgggag |
| | | gcacagtcaccatcaagtgccaggccagtcagaacatttacagcaatttag |
| | | cctggtatcagcagaaaccagggcagcctcccaagcgcctgatctacaa |
| | | ggcatccactctgccatctggggtcccatcgcggttcaaaggcagtggat |
| | | ctgggacacagttcactctcaccatcagcgacctggagtgtgccgatgctg |
| | | ccacttactactgtcaaagcaattattatagtagtagtagtaattatggcccta |
| | | atgctttcggcggagggaccgaggtggtcgtcaaa |
| 16K-LC-DNA | (SEQ ID NO: 130) | gatgtcgtgatgacccagactccagcctccgtgtctgaacctgtgggagg |
| | | cacagtcaccatcaattgccaggccattcagagcattggtaataattttgtct |
| | | ggtatcagcagaaaccagggcagcctcccaagctcctgatctacaaggc |
| | | atccattcgggcatctggggtctcatcgcggttcaggggcagtggatctgg |
| | | gacagacttcactctcaccatcagcgacctggagtgtgacgatactggca |
| | | cttactactgtcaatgtacttatgctagtagtgctactttcggcggagggacc |
| | | gaggtggtcgtcaaa |
| 22K-LC-DNA | (SEQ ID NO: 131) | gaccctgtgctgacccagactccatcgtccgtgtctgcagctgtgggagg |
| | | cacagtcaccatcagttgccaggccactgagactgtttatagtaacgaccg |
| | | cttagcctggtatcagcagaaaccagggcagcctcccaagctcctgatcta |
| | | caggatatccactctggaatctggggtcccatcgcggttcaaaggcagtg |
| | | gatctgggacacagttcactctcaccatcagcggcgtgcagtgtgacgat |
| | | gctgccacttactactgtgcaggcggttataatggtaatatttgggctttcgg |
| | | cggagggaccgaggtggtcgtcaaa |
| 26K-LC-DNA | (SEQ ID NO: 132) | gatgtcgtgatgacccagactgcatcccccgtgtctggagctgtgggagg |
| | | cacagtcaccatcaagtgccaggccagtgaggacatttacagctctttagc |
| | | ctggtatcagcagaaaccagggcagcctcccaagctcctgatctaccagg |
| | | catccaaactggcatctggggtcccaccgcggttcagcggcagtggatct |
| | | gggacagagtacactctcaccatcagcgacctggagtgtgccgatgctgc |
| | | cacttactactgtcagaactattattatgttcgtggtgctgattattgggctttc |
| | | ggcggagggaccgaggtggtcgtcgaa |

The embodiments anti-TREM2 antibodies were specifically screened for candidates that do not block TREM2-ligand interactions (e.g., ligands such as phospholipids and oAβ). Phospholipid-TREM2 and oAβ-TREM2 interactions have been shown to play crucial roles in microglia survival, apoptosis, depolarization, cytokine expression, and clustering around amyloid plaques (Zhao, Y., et al., TREM2Is a Receptor for f-Amyloid that Mediates Microglial Function. Neuron, 2018. 97(5): p. 1023-1031.e7; Wang, Y., et al., TREM2 lipid sensing sustains the microglial response in an Alzheimer's disease model. Cell, 2015. 160(6): p. 1061-71). TREM2 agonistic antibodies reported in previous publications were not characterized whether they would affect the ligand-TREM2 interactions (Schlepckow, K., et al., Enhancing protective microglial activities with a dual function TREM2 antibody to the stalk region. EMBO Mol Med, 2020. 12(4): p. e1227; Cheng, Q., et al., TREM2-activating antibodies abrogate the negative pleiotropic effects of the Alzheimer's disease variant Trem2(R47H) on murine myeloid cell function. J Biol Chem, 2018. 293(32): p. 12620-12633; Wang, S., et al., Anti-human TREM2 induces microglia proliferation and reduces pathology in an Alzheimer's disease model. J Exp Med, 2020. 217(9); Fassler, M., et al., Engagement of TREM2 by a novel monoclonal antibody induces activation of microglia and improves cognitive function in Alzheimer's disease models. Journal of Neuroinflammation, 2021. 18(1): p. 19; Price, B. R., et al., Therapeutic Trem2 activation ameliorates amyloid-beta deposition and improves cognition in the 5×FAD model of amyloid deposition. Journal of Neuroinflammation, 2020. 17(1): p. 238).

In some embodiments, an antibody of the present disclosure (e.g., Ab18) can activate TREM2 as a soluble antibody without the need for solid surface coating or engaging Fc receptors.

The present disclosure shows that Ab18 TVD-Ig and the Ab18 IgG formats stimulated the phagocytosis of oAβ-lipid. Amyloid plaques are naturally associated with lipids and APOE (Kiskis, J., et al., Plaque-associated lipids in Alzheimer's disease brain tissue visualized by nonlinear microscopy. Scientific Reports, 2015. 5(1): p. 13489; Parhizkar, S., et al., Loss of TREM2 function increases amyloid seeding but reduces plaque-associated ApoE. Nature Neuroscience, 2019. 22(2): p. 191-204; Liao, C. R., et al., Synchrotron F77R reveals lipid around and within amyloid plaques in transgenic mice and Alzheimer's disease brain. Analyst, 2013. 138(14): p. 3991-3997; Namba, Y., et al., Apolipoprotein E immunoreactivity in cerebral amyloid deposits and neurofibrillary tangles in Alzheimer's disease and kuru plaque amyloid in Creutzfeldt-Jakob disease. Brain Res, 1991.541(1): p. 163-6; Xiong, F., W. Ge, and C. Ma, Quantitative proteomics reveals distinct composition of amyloid plaques in Alzheimer's disease. Alzheimers Dement, 2019. 15(3): p. 429-440). Lipid interactions with Aβ fibrils contribute to the formation of more neurotoxic protofibrils (Liao, C. R., et al., Synchrotron FTIR reveals lipid around and within amyloid plaques in transgenic mice and Alzheimer's disease brain. Analyst, 2013. 138(14): p. 3991-3997; Martins, I. C., et al., Lipids revert inert Abeta amyloid fibrils to neurotoxic protofibrils that affect learning in mice. Embo j, 2008. 27(1): p. 224-33). APOE is associated with amyloid plaques, helps plaque seeding, and affects plaque clearance (Parhizkar, S., et al., Loss of TREM2function increases amyloid seeding but reduces plaque-associated ApoE. Nature Neuroscience, 2019. 22(2): p. 191-204; Castellano, J. M., et al., Human apoE isoforms differentially regulate brain amyloid-f peptide clearance. Sci Transl Med, 2011. 3(89): p. 89ra57; Liu, C. C., et al., Apolipoprotein E and Alzheimer disease: risk, mechanisms and therapy. Nat Rev Neurol, 2013. 9(2): p. 106-18; Liu, C. C., et al., ApoE4 Accelerates Early Seeding of Amyloid Pathology. Neuron, 2017. 96(5): p. 1024-1032.e3; Spangenberg, E., et al., Sustained microglial depletion with CSF1R inhibitor impairs parenchymal plaque development in an Alzheimer's disease model. Nature Communications, 2019. 10(1): p. 3758).

When the valency of Ab18 was increased to tetravalent, a 100-fold increased TREM2 activation was observed. The increased activation was manifested by the stronger biological effects, both in vitro and in vivo. The ITAM signaling pathway involves a multivalent ligand to induce receptor clustering and trigger the downstream signaling cascade.

Delivering Ab18 into the brain by targeting TfR using a bispecific antibody increased antibody brain concentration by more than 10-fold. The increased antibody brain entry was manifested as in vivo beneficial effects in ameliorating amyloid plaque pathology.

In some embodiments, the inventors observed enhanced microglia clustering around plaques with increased phagocytosis of plaques by Ab18 treatment.

Ab18/αTfR treatment showed reduced neuron damage indicated by the significant reduction of plaque-proximal dystrophic neurites in both number and staining intensity. Although TREM2 agonism increased the phagocytosis of lipid-oAβ, this did not cause bystander detrimental damages to neurons. The positive connection between TREM2 agonism and clearance of apoptotic neurons is consistent with the utility of such antibody constructs in the treatment of neurodegenerative diseases and disorders including, but, not limited to Alzheimer's Disease (AD), Parkinson's Disease (PD), dementia, dementia with Lewy bodies (DLB) and others, including neuroinflammatory processes and those involving microglia.

The embodiments described above are presented by way of example only and are not intended as a limitation upon the concepts and principles of the present disclosure. As such, it will be appreciated by one having ordinary skill in the art that various changes in the elements and their configuration and arrangement are possible without departing from the spirit and scope of the present disclosure. Various features and aspects of the present disclosure are set forth in the following embodiments.

### Exemplary Embodiments

1. An isolated bispecific antibody that specifically binds to TREM2, the antibody comprising:
a first portion that includes a plurality of first-specificity binding sites, each of the first-specificity binding sites being capable of binding to TREM2; and
a second portion that includes a second-specificity binding site capable of binding to TfR.
   2. The bispecific antibody of Embodiment 1, wherein the plurality comprises four of the first-specificity binding sites.
   3. An isolated monoclonal antibody, wherein the antibody specifically binds to TREM2 and wherein the antibody competes for binding of the TREM2 epitopes with an antibody selected from the group consisting of TREM2-Ab1Rb, TREM2-Ab2Rb, TREM2-Ab6Rb, TREM2-Ab12Rb, TREM2-Ab16Rb, TREM2-Ab22Rb, TREM2-Ab26Rb, TREM2-Ab2HuRb, and TREM2-Ab8HuRb, and TREM2-Ab19HuRb.
   4. The antibody of any one of the preceding Embodiments, wherein the antibody comprises:
(a) a first V_{H} CDR at least 80% identical to 1H-HCDR1-AA (SEQ ID NO: 1), 2H-HCDR1-AA (SEQ ID NO: 4), 12H-HCDR1-AA (SEQ ID NO: 7), 16H-HCDR1-AA (SEQ ID NO: 10), 22H-HCDR1-AA (SEQ ID NO: 13), 26H-HCDR1-AA (SEQ ID NO: 16), H2-Hu-HCDR1-AA (SEQ ID NO: 19), H8-Hu-HCDR1-AA (SEQ ID NO: 22), or H19-Hu-HCDR1-AA (SEQ ID NO: 25);
(b) a second V_{H} CDR at least 80% identical to 1H-HCDR2-AA (SEQ ID NO: 2), 2H-HCDR2-AA (SEQ ID NO: 5), 12H-HCDR2-AA (SEQ ID NO: 8), 16H-HCDR2-AA (SEQ ID NO: 11), 22H-HCDR2-AA (SEQ ID NO: 14), 26H-HCDR2-AA (SEQ ID NO: 17), H2-Hu-HCDR2-AA (SEQ ID NO: 20), H8-Hu-HCDR2-AA (SEQ ID NO: 23), or H19-Hu-HCDR2-AA (SEQ ID NO: 26);
(c) a third V_{H} CDR at least 80% identical to 1H-HCDR3-AA (SEQ ID NO: 3), 2H-HCDR3-AA (SEQ ID NO: 6), 12H-HCDR3-AA (SEQ ID NO: 9), 16H-HCDR3-AA (SEQ ID NO: 12), 22H-HCDR3-AA (SEQ ID NO: 15), 26H-HCDR3-AA (SEQ ID NO: 18), H2-Hu-HCDR3-AA (SEQ ID NO: 21), H8-Hu-HCDR3-AA (SEQ ID NO: 24), or H19-Hu-HCDR3-AA (SEQ ID NO: 27);
(d) a first V_{L} region at least 80% identical to 1K-LCDR1-AA (SEQ ID NO: 28), 2K-LCDR1-AA (SEQ ID NO: 30), 6K-LCDR1-AA (SEQ ID NO: 32), 12K-LCDR1-AA (SEQ ID NO: 34), 16K-LCDR1-AA (SEQ ID NO: 36), 22K-LCDR1-AA (SEQ ID NO: 38), 26K-LCDR1-AA (SEQ ID NO: 40), 2L-Hu-LCDR1-AA (SEQ ID NO: 42), 8L-Hu-LCDR1-AA (SEQ ID NO: 44), or 19L-Hu-LCDR1-AA (SEQ ID NO: 46);
(e) a second V_{L} CDR at least 80% identical to 1K-LCDR2-AA (tripeptide GAS), 2K-LCDR2-AA (tripeptide GAS), 6K-LCDR2-AA (tripeptide GAS), 12K-LCDR2-AA (tripeptide KAS), 16K-LCDR2-AA (tripeptide KAS), 22K-LCDR2-AA (tripeptide RIS), 26K-LCDR2-AA (tripeptide QAS), 2L-Hu-LCDR2-AA (tripeptide EVS), 8L-Hu-LCDR2-AA (tripeptide TNN), or 19L-Hu-LCDR2-AA (tripeptide DVT); and
(f) a third V_{L} CDR at least 80% identical to 1K-LCDR3-AA (SEQ ID NO: 29), 2K-LCDR3-AA (SEQ ID NO: 31), 6K-LCDR3-AA (SEQ ID NO: 33), 12K-LCDR3-AA (SEQ ID NO: 35), 16K-LCDR3-AA (SEQ ID NO: 37), 22K-LCDR3-AA (SEQ ID NO: 39), 26K-LCDR3-AA (SEQ ID NO: 41), 2L-Hu-LCDR3-AA (SEQ ID NO: 43), B09 (SEQ ID NO: 27), 8L-Hu-LCDR3-AA (SEQ ID NO: 45), or 19L-Hu-LCDR3-AA (SEQ ID NO: 47);
   5. The isolated antibody of Embodiment 4, wherein the antibody comprises:
(a) a first V_{H} CDR is identical to SEQ ID NO: 1;
(b) a second portion that includes V_{H} CDR is identical to SEQ ID NO: 2;
(c) a third V_{H} CDR is identical to SEQ ID NO: 3;
(d) a first V_{L} CDR is identical to SEQ ID NO: 28;
(e) a second V_{L} CDR is identical to tripeptide GAS; and
(f) a third V_{L} CDR is identical to SEQ ID NO: 29.
   6. The isolated antibody of Embodiment 4, wherein the antibody comprises:
(a) a first V_{H} CDR is identical to SEQ ID NO: 4;
(b) a second V_{H} CDR is identical to SEQ ID NO: 5;
(c) a third V_{H} CDR is identical to SEQ ID NO: 6;
(d) a first V_{L} CDR is identical to SEQ ID NO: 30;
(e) a second V_{L} CDR is identical to tripeptide GAS; and
(f) a third V_{L} CDR is identical to SEQ ID NO: 31.
   7. The isolated antibody of Embodiment 4, wherein the antibody comprises:
(a) a first V_{H} CDR is identical to SEQ ID NO: 7;
(b) a second V_{H} CDR is identical to SEQ ID NO: 8;
(c) a third V_{H} CDR is identical to SEQ ID NO: 9;
(d) a first V_{L} CDR is identical to SEQ ID NO: 32;
(e) a second V_{L} CDR is identical to tripeptide GAS; and
(f) a third V_{L} CDR is identical to SEQ ID NO: 33.
   8. The isolated antibody of Embodiment 4, wherein the antibody comprises:
(a) a first V_{H} CDR is identical to SEQ ID NO: 10;
(b) a second V_{H} CDR is identical to SEQ ID NO: 11;
(c) a third V_{H} CDR is identical to SEQ ID NO: 12;
(d) a first V_{L} CDR is identical to SEQ ID NO: 34;
(e) a second V_{L} CDR is identical to tripeptide KAS; and
(f) a third V_{L} CDR is identical to SEQ ID NO: 35.
   9. The isolated antibody of Embodiment 4, wherein the antibody comprises:
(a) a first V_{H} CDR is identical to SEQ ID NO: 13;
(b) a second V_{H} CDR is identical to SEQ ID NO: 14;
(c) a third V_{H} CDR is identical to SEQ ID NO: 15;
(d) a first V_{L} CDR is identical to SEQ ID NO: 36;
(e) a second V_{L} CDR is identical to tripeptide KAS; and
(f) a third V_{L} CDR is identical to SEQ ID NO: 37.
   10. The isolated antibody of Embodiment 4, wherein the antibody comprises:
(a) a first V_{H} CDR is identical to SEQ ID NO: 16;
(b) a second V_{H} CDR is identical to SEQ ID NO: 17;
(c) a third V_{H} CDR is identical to SEQ ID NO: 18;
(d) a first V_{L} CDR is identical to SEQ ID NO: 38;
(e) a second V_{L} CDR is identical to tripeptide RIS; and
(f) a third V_{L} CDR is identical to SEQ ID NO: 39.
   11. The isolated antibody of Embodiment 4, wherein the antibody comprises:
(a) a first V_{H} CDR is identical to SEQ ID NO: 19;
(b) a second V_{H} CDR is identical to SEQ ID NO: 20;
(c) a third V_{H} CDR is identical to SEQ ID NO: 21;
(d) a first V_{L} CDR is identical to SEQ ID NO: 40;
(e) a second V_{L} CDR is identical to tripeptide QAS; and
(f) a third V_{L} CDR is identical to SEQ ID NO: 41.
   12. The isolated antibody of Embodiment 4, wherein the antibody comprises:
(a) a first V_{H} CDR is identical to SEQ ID NO: 22;
(b) a second V_{H} CDR is identical to SEQ ID NO: 23;
(c) a third V_{H} CDR is identical to SEQ ID NO: 24;
(d) a first V_{L} CDR is identical to SEQ ID NO: 42;
(e) a second V_{L} CDR is identical to tripeptide EVS; and
(f) a third V_{L} CDR is identical to SEQ ID NO: 43.
   13. The isolated antibody of Embodiment 4, wherein the antibody comprises:
(a) a first V_{H} CDR is identical to SEQ ID NO: 25;
(b) a second V_{H} CDR is identical to SEQ ID NO: 26;
(c) a third V_{H} CDR is identical to SEQ ID NO: 27;
(d) a first V_{L} CDR is identical to SEQ ID NO: 44;
(e) a second V_{L} CDR is identical to tripeptide TNN; and
(f) a third V_{L} CDR is identical to SEQ ID NO: 45.
   14. The antibody of Embodiment 4, wherein the antibody comprises:
(i) a V_{H} domain at least about 80% identical to the V_{H} domain of 1H-HC-AA (SEQ ID NO: 61) or the humanized V_{H} domain of 1H-HC-AA; and a V_{L} domain at least about 80% identical to the V_{L} domain of 1K-LC-AA (SEQ ID NO: 67) or the humanized V_{L} domain of 1K-LC-AA;
(ii) a V_{H} domain at least about 80% identical to the V_{H} domain of 2H-HC-AA (SEQ ID NO: 62) or the humanized V_{H} domain of 2H-HC-AA; and a V_{L} domain at least about 80% identical to the V_{L} domain of 2K-LC-AA (SEQ ID NO: 68) or the humanized V_{L} domain of 2K-LC-AA;
(iii) a V_{H} domain at least about 80% identical to the V_{H} domain of 2H-HC-AA (SEQ ID NO: 62) or the humanized V_{H} domain of 2H-HC-AA; and a V_{L} domain at least about 80% identical to the V_{L} domain of 6K LC-AA (SEQ ID NO: 69) or the humanized V_{L} domain of 6K LC-AA;
(iv) a V_{H} domain at least about 80% identical to the V_{H} domain of 12H-HC-AA (SEQ ID NO: 63) or the humanized V_{H} domain of 12H-HC-AA; and a V_{L} domain at least about 80% identical to the V_{L} domain of 12K-LC-AA (SEQ ID NO: 70) or the humanized V_{L} domain of 12K-LC-AA;
(v) a V_{H} domain at least about 80% identical to the V_{H} domain of 16H-HC-AA (SEQ ID NO: 64) or the humanized V_{H} domain of 16H-HC-AA; and a V_{L} domain at least about 80% identical to the V_{L} domain of 16K-LC-AA (SEQ ID NO: 71) or the humanized V_{L} domain of 16K-LC-AA;
(vi) a V_{H} domain at least about 80% identical to the V_{H} domain of 22H-HC-AA (SEQ ID NO: 65) or the humanized V_{H} domain of 22H-HC-AA; and a V_{L} domain at least about 80% identical to the V_{L} domain of 22K-LC-AA (SEQ ID NO: 72) or the humanized V_{L} domain of 22K-LC-AA;
(vii) a V_{H} domain at least about 80% identical to the V_{H} domain of 26H-HC-AA (SEQ ID NO: 66) or the humanized V_{H} domain of 26H-HC-AA; and a V_{L} domain at least about 80% identical to the V_{L} domain of 26K-LC-AA (SEQ ID NO: 73) or the humanized V_{L} domain of 26K-LC-AA;
(viii) a V_{H} domain at least about 80% identical to the V_{H} domain of H2-Hu-HC-AA (SEQ ID NO: 58) or the humanized V_{H} domain of H2-Hu-HC-AA; and a V_{L} domain at least about 80% identical to the V_{L} domain of 2L-Hu-LC-AA (SEQ ID NO: 74) or the humanized V_{L} domain of 2L-Hu-LC-AA;
(ix) a V_{H} domain at least about 80% identical to the V_{H} domain of H8-Hu-HC-AA (SEQ ID NO: 59) or the humanized V_{H} domain of H8-Hu-HC-AA; and a V_{L} domain at least about 80% identical to the V_{L} domain of 8L-Hu-LC-AA (SEQ ID NO: 75) or the humanized V_{L} domain of 8L-Hu-LC-AA; or
(x) a V_{H} domain at least about 80% identical to the V_{H} domain of H19-Hu HC-AA (SEQ ID NO: 66) or the humanized V_{H} domain of H19-Hu HC-AA; and a V_{L} domain at least about 80% identical to the V_{L} domain of 19L-Hu-LC-AA (SEQ ID NO: 76) or the humanized V_{L} domain of 19L-Hu-LC-AA.
   15. The antibody of any one of Embodiments 1-14, wherein the antibody is recombinant.
   16. The antibody of any one of Embodiments 1-14, wherein the antibody is an IgG, IgM, IgA or an antigen binding fragment thereof.
   17. The antibody of any one of Embodiments 1-14, wherein the antibody is a Fab′, a F(ab′)2, a F(ab′)3, a monovalent second-specificity binding site.
   18. The antibody of any one of Embodiments 1-14, wherein the antibody is a human, humanized antibody or de-immunized antibody.
   19. The antibody of any one of Embodiments 1-14, wherein the antibody is conjugated to an imaging agent.
   20. A chimeric antigen receptor comprising an antigen-binding domain at least 80% identical to an antigen-binding domain of the monoclonal antibody of any one of the preceding Embodiments.
   21. A composition comprising an antibody of any one of Embodiments 1-14 in a pharmaceutically acceptable carrier.
   22. An isolated polynucleotide molecule comprising a nucleic acid sequence encoding an antibody of any one of Embodiments 1-14. 23. A recombinant polypeptide comprising an antibody V_{H} domain comprising CDRs 1-3 of the V_{H} domain of TREM2-Ab1Rb (SEQ ID NOs: 1, 2, and 3); CDRs 1-3 of the V_{H} domain of TREM2-Ab2Rb (SEQ ID NOs: 4, 5, and 6); CDRs 1-3 of the V_{H} domain of TREM2-Ab2Rb (SEQ ID NOs: 4, 5, and 6); CDRs 1-3 of the V_{H} domain of TREM2-Ab12Rb (SEQ ID NOs: 7, 8, and 9); CDRs 1-3 of the V_{H} domain of TREM2-Ab16Rb (SEQ ID NOs: 10, 11, and 12); CDRs 1-3 of the V_{H} domain of TREM2-Ab22Rb (SEQ ID NOs: 13, 14, and 15); CDRs 1-3 of the V_{H} domain of TREM2-Ab26Rb (SEQ ID NOs: 16, 17, and 18); CDRs 1-3 of the V_{H} domain of TREM2-Ab2Hu (SEQ ID NOs: 19, 20, and 21); CDRs 1-3 of the V_{H} domain of TREM2-Ab8Hu (SEQ ID NOs: 22, 23, and 24); or CDRs 1-3 of the V_{H} domain of TREM2-Ab119Hu (SEQ ID NOs: 25, 26, and 27).
   24. A recombinant polypeptide comprising an antibody V_{L} domain comprising CDRs 1-3 of the V_{L} domain of TREM2-Ab1Rb; CDRs 1-3 of the V_{L} domain of TREM2-Ab2Rb; CDRs 1-3 of the V_{L} domain of TREM2-Ab6Rb; CDRs 1-3 of the V_{L} domain of TREM2-Ab12Rb; CDRs 1-3 of the V_{L} domain of TREM2-Ab16Rb; CDRs 1-3 of the V_{L} domain of TREM2-Ab22Rb; CDRs 1-3 of the V_{L} domain of TREM2-Ab26Rb; CDRs 1-3 of the V_{L} domain of TREM2-Ab2Hu; CDRs 1-3 of the V_{L} domain of TREM2-Ab8Hu; or CDRs 1-3 of the V_{L} domain of TREM2-Ab19Hu.
   25. An isolated polynucleotide molecule comprising a nucleic acid sequence encoding a polypeptide of Embodiment 23 or 24.
   26. A host cell comprising one or more polynucleotide molecule(s) encoding an antibody of any one of Embodiments 1-14 or a recombinant polypeptide of Embodiment 23 or 24.
   27. The host cell of Embodiment 26, wherein the host cell is a mammalian cell, a yeast cell, a bacterial cell, a ciliate cell or an insect cell.
   28. An expression vector comprising a polynucleotide having at least 95% identity to H2-Hu-HC-DNA, H8-Hu-HC-DNA, 19H-Hu HC-DNA, 16H-HC-DNA, 22H-HC-DNA, 26H-HC-DNA, H2-Hu-HC-DNA, H8-Hu-HC-DNA, or H19-Hu-HC-DNA.
   29. An expression vector comprising a polynucleotide having at least 95% identity to 20L-LC-DNA, 8L-LC-DNA, 19L LC-DNA, 1K-LC-DNA. 2K-LC-DNA, 6K-LC-DNA, 2K-LC-DNA, 16K-LC-DNA, 22K-LC-DNA, or 26K-LC-DNA.
   30. A method of manufacturing an antibody comprising:
(a) expressing one or more polynucleotide molecule(s) encoding a V_{L} and V_{H} chain of an antibody of any one of Embodiments 1-14 in a cell; and
(b) purifying the antibody from the cell and/or a fluid medium in which the cell is disposed.

What is claimed is:

1. An isolated bispecific antibody that specifically binds to TREM2, the antibody comprising:
a first portion that includes a plurality of first-specificity binding sites, each of the first-specificity binding sites being capable of binding to TREM2; and
a second portion that includes a second-specificity binding site capable of binding to TfR.

2. The bispecific antibody of claim 1, wherein the plurality comprises four of the first-specificity binding sites.

3. An isolated monoclonal antibody, wherein the antibody specifically binds to TREM2 and wherein the antibody competes for binding of the TREM2 epitopes with an antibody selected from the group consisting of TREM2-Ab1Rb, TREM2-Ab2Rb, TREM2-Ab6Rb, TREM2-Ab12Rb, TREM2-Ab16Rb, TREM2-Ab22Rb, TREM2-Ab26Rb, TREM2-Ab2HuRb, and TREM2-Ab8HuRb, and TREM2-Ab19HuRb.

4. The antibody of any one of the preceding claims, wherein the antibody comprises:
(a) a first V_{H} CDR at least 80% identical to 1H-HCDR1-AA (SEQ ID NO: 1), 2H-HCDR1-AA (SEQ ID NO: 4), 12H-HCDR1-AA (SEQ ID NO: 7), 16H-HCDR1-AA (SEQ ID NO: 10), 22H-HCDR1-AA (SEQ ID NO: 13), 26H-HCDR1-AA (SEQ ID NO: 16), H2-Hu-HCDR1-AA (SEQ ID NO: 19), H8-Hu-HCDR1-AA (SEQ ID NO: 22), or H19-Hu-HCDR1-AA (SEQ ID NO: 25);
(b) a second V_{H} CDR at least 80% identical to 1H-HCDR2-AA (SEQ ID NO: 2), 2H-HCDR2-AA (SEQ ID NO: 5), 12H-HCDR2-AA (SEQ ID NO: 8), 16H-HCDR2-AA (SEQ ID NO: 11), 22H-HCDR2-AA (SEQ ID NO: 14), 26H-HCDR2-AA (SEQ ID NO: 17), H2-Hu-HCDR2-AA (SEQ ID NO: 20), H8-Hu-HCDR2-AA (SEQ ID NO: 23), or H19-Hu-HCDR2-AA (SEQ ID NO: 26);
(c) a third V_{H} CDR at least 80% identical to 1H-HCDR3-AA (SEQ ID NO: 3), 2H-HCDR3-AA (SEQ ID NO: 6), 12H-HCDR3-AA (SEQ ID NO: 9), 16H-HCDR3-AA (SEQ ID NO: 12), 22H-HCDR3-AA (SEQ ID NO: 15), 26H-HCDR3-AA (SEQ ID NO: 18), H2-Hu-HCDR3-AA (SEQ ID NO: 21), H8-Hu-HCDR3-AA (SEQ ID NO: 24), or H19-Hu-HCDR3-AA (SEQ ID NO: 27);
(d) a first V_{L} region at least 80% identical to 1K-LCDR1-AA (SEQ ID NO: 28), 2K-LCDR1-AA (SEQ ID NO: 30), 6K-LCDR1-AA (SEQ ID NO: 32), 12K-LCDR1-AA (SEQ ID NO: 34), 16K-LCDR1-AA (SEQ ID NO: 36), 22K-LCDR1-AA (SEQ ID NO: 38), 26K-LCDR1-AA (SEQ ID NO: 40), 2L-Hu-LCDR1-AA (SEQ ID NO: 42), 8L-Hu-LCDR1-AA (SEQ ID NO: 44), or 19L-Hu-LCDR1-AA (SEQ ID NO: 46);
(e) a second V_{L} CDR at least 80% identical to 1K-LCDR2-AA (tripeptide GAS), 2K-LCDR2-AA (tripeptide GAS), 6K-LCDR2-AA (tripeptide GAS), 12K-LCDR2-AA (tripeptide KAS), 16K-LCDR2-AA (tripeptide KAS), 22K-LCDR2-AA (tripeptide RIS), 26K-LCDR2-AA (tripeptide QAS), 2L-Hu-LCDR2-AA (tripeptide EVS), 8L-Hu-LCDR2-AA (tripeptide TNN), or 19L-Hu-LCDR2-AA (tripeptide DVT); and
(f) a third V_{L} CDR at least 80% identical to 1K-LCDR3-AA (SEQ ID NO: 29), 2K-LCDR3-AA (SEQ ID NO: 31), 6K-LCDR3-AA (SEQ ID NO: 33), 12K-LCDR3-AA (SEQ ID NO: 35), 16K-LCDR3-AA (SEQ ID NO: 37), 22K-LCDR3-AA (SEQ ID NO: 39), 26K-LCDR3-AA (SEQ ID NO: 41), 2L-Hu-LCDR3-AA (SEQ ID NO: 43), B09 (SEQ ID NO: 27), 8L-Hu-LCDR3-AA (SEQ ID NO: 45), or 19L-Hu-LCDR3-AA (SEQ ID NO: 47);

5. The isolated antibody of claim 4, wherein the antibody comprises:
(a) a first V_{H} CDR is identical to SEQ ID NO: 1;
(b) a second portion that includes V_{H} CDR is identical to SEQ ID NO: 2;
(c) a third V_{H} CDR is identical to SEQ ID NO: 3;
(d) a first V_{L} CDR is identical to SEQ ID NO: 28;
(e) a second V_{L} CDR is identical to tripeptide GAS; and
(f) a third V_{L} CDR is identical to SEQ ID NO: 29.

6. The isolated antibody of claim 4, wherein the antibody comprises:
(a) a first V_{H} CDR is identical to SEQ ID NO: 4;
(b) a second V_{H} CDR is identical to SEQ ID NO: 5;
(c) a third V_{H} CDR is identical to SEQ ID NO: 6;
(d) a first V_{L} CDR is identical to SEQ ID NO: 30;
(e) a second V_{L} CDR is identical to tripeptide GAS; and
(f) a third V_{L} CDR is identical to SEQ ID NO: 31.

7. The isolated antibody of claim 4, wherein the antibody comprises:
(a) a first V_{H} CDR is identical to SEQ ID NO: 7;
(b) a second V_{H} CDR is identical to SEQ ID NO: 8;
(c) a third V_{H} CDR is identical to SEQ ID NO: 9;
(d) a first V_{L} CDR is identical to SEQ ID NO: 32;
(e) a second V_{L} CDR is identical to tripeptide GAS; and
(f) a third V_{L} CDR is identical to SEQ ID NO: 33.

8. The isolated antibody of claim 4, wherein the antibody comprises:
(a) a first V_{H} CDR is identical to SEQ ID NO: 10;
(b) a second V_{H} CDR is identical to SEQ ID NO: 11;
(c) a third V_{H} CDR is identical to SEQ ID NO: 12;
(d) a first V_{L} CDR is identical to SEQ ID NO: 34;
(e) a second V_{L} CDR is identical to tripeptide KAS; and
(f) a third V_{L} CDR is identical to SEQ ID NO: 35.

9. The isolated antibody of claim 4, wherein the antibody comprises:
(a) a first V_{H} CDR is identical to SEQ ID NO: 13;
(b) a second V_{H} CDR is identical to SEQ ID NO: 14;
(c) a third V_{H} CDR is identical to SEQ ID NO: 15;
(d) a first V_{L} CDR is identical to SEQ ID NO: 36;
(e) a second V_{L} CDR is identical to tripeptide KAS; and
(f) a third V_{L} CDR is identical to SEQ ID NO: 37.

10. The isolated antibody of claim 4, wherein the antibody comprises:
(a) a first V_{H} CDR is identical to SEQ ID NO: 16;
(b) a second V_{H} CDR is identical to SEQ ID NO: 17;
(c) a third V_{H} CDR is identical to SEQ ID NO: 18;
(d) a first V_{L} CDR is identical to SEQ ID NO: 38;
(e) a second V_{L} CDR is identical to tripeptide RIS; and
(f) a third V_{L} CDR is identical to SEQ ID NO: 39.

11. The isolated antibody of claim 4, wherein the antibody comprises:
(a) a first V_{H} CDR is identical to SEQ ID NO: 19;
(b) a second V_{H} CDR is identical to SEQ ID NO: 20;
(c) a third V_{H} CDR is identical to SEQ ID NO: 21;
(d) a first V_{L} CDR is identical to SEQ ID NO: 40;
(e) a second V_{L} CDR is identical to tripeptide QAS; and
(f) a third V_{L} CDR is identical to SEQ ID NO: 41.

12. The isolated antibody of claim 4, wherein the antibody comprises:
(a) a first V_{H} CDR is identical to SEQ ID NO: 22;
(b) a second V_{H} CDR is identical to SEQ ID NO: 23;
(c) a third V_{H} CDR is identical to SEQ ID NO: 24;
(d) a first V_{L} CDR is identical to SEQ ID NO: 42;
(e) a second V_{L} CDR is identical to tripeptide EVS; and
(f) a third V_{L} CDR is identical to SEQ ID NO: 43.

13. The isolated antibody of claim 4, wherein the antibody comprises:
(a) a first V_{H} CDR is identical to SEQ ID NO: 25;
(b) a second V_{H} CDR is identical to SEQ ID NO: 26;
(c) a third V_{H} CDR is identical to SEQ ID NO: 27;
(d) a first V_{L} CDR is identical to SEQ ID NO: 44;
(e) a second V_{L} CDR is identical to tripeptide TNN; and
(f) a third V_{L} CDR is identical to SEQ ID NO: 45.

14. The antibody of claim 4, wherein the antibody comprises:
(i) a V_{H} domain at least about 80% identical to the V_{H} domain of 1H-HC-AA (SEQ ID NO: 61) or the humanized V_{H} domain of 1H-HC-AA; and a V_{L} domain at least about 80% identical to the V_{L} domain of 1K-LC-AA (SEQ ID NO: 67) or the humanized V_{L} domain of 1K-LC-AA;
(ii) a V_{H} domain at least about 80% identical to the V_{H} domain of 2H-HC-AA (SEQ ID NO: 62) or the humanized V_{H} domain of 2H-HC-AA; and a V_{L} domain at least about 80% identical to the V_{L} domain of 2K-LC-AA (SEQ ID NO: 68) or the humanized V_{L} domain of 2K-LC-AA;
(iii) a V_{H} domain at least about 80% identical to the V_{H} domain of 2H-HC-AA (SEQ ID NO: 62) or the humanized V_{H} domain of 2H-HC-AA; and a V_{L} domain at least about 80% identical to the V_{L} domain of 6K LC-AA (SEQ ID NO: 69) or the humanized V_{L} domain of 6K LC-AA;
(iv) a V_{H} domain at least about 80% identical to the V_{H} domain of 12H-HC-AA (SEQ ID NO: 63) or the humanized V_{H} domain of 12H-HC-AA; and a V_{L} domain at least about 80% identical to the V_{L} domain of 12K-LC-AA (SEQ ID NO: 70) or the humanized V_{L} domain of 12K-LC-AA;
(v) a V_{H} domain at least about 80% identical to the V_{H} domain of 16H-HC-AA (SEQ ID NO: 64) or the humanized V_{H} domain of 16H-HC-AA; and a V_{L} domain at least about 80% identical to the V_{L} domain of 16K-LC-AA (SEQ ID NO: 71) or the humanized V_{L} domain of 16K-LC-AA;
(vi) a V_{H} domain at least about 80% identical to the V_{H} domain of 22H-HC-AA (SEQ ID NO: 65) or the humanized V_{H} domain of 22H-HC-AA; and a V_{L} domain at least about 80% identical to the V_{L} domain of 22K-LC-AA (SEQ ID NO: 72) or the humanized V_{L} domain of 22K-LC-AA;
(vii) a V_{H} domain at least about 80% identical to the V_{H} domain of 26H-HC-AA (SEQ ID NO: 66) or the humanized V_{H} domain of 26H-HC-AA; and a V_{L} domain at least about 80% identical to the V_{L} domain of 26K-LC-AA (SEQ ID NO: 73) or the humanized V_{L} domain of 26K-LC-AA;
(viii) a V_{H} domain at least about 80% identical to the V_{H} domain of H2-Hu-HC-AA (SEQ ID NO: 58) or the humanized V_{H} domain of H2-Hu-HC-AA; and a V_{L} domain at least about 80% identical to the V_{L} domain of 2L-Hu-LC-AA (SEQ ID NO: 74) or the humanized V_{L} domain of 2L-Hu-LC-AA;
(ix) a V_{H} domain at least about 80% identical to the V_{H} domain of H8-Hu-HC-AA (SEQ ID NO: 59) or the humanized V_{H} domain of H8-Hu-HC-AA; and a V_{L} domain at least about 80% identical to the V_{L} domain of 8L-Hu-LC-AA (SEQ ID NO: 75) or the humanized V_{L} domain of 8L-Hu-LC-AA; or
(x) a V_{H} domain at least about 80% identical to the V_{H} domain of H19-Hu HC-AA (SEQ ID NO: 66) or the humanized V_{H} domain of H19-Hu HC-AA; and a V_{L} domain at least about 80% identical to the V_{L} domain of 19L-Hu-LC-AA (SEQ ID NO: 76) or the humanized V_{L} domain of 19L-Hu-LC-AA.

15. The antibody of any one of claims 1-14, wherein the antibody is recombinant.

16. The antibody of any one of claims 1-14, wherein the antibody is an IgG, IgM, IgA or an antigen binding fragment thereof.

17. The antibody of any one of claims 1-14, wherein the antibody is a Fab′, a F(ab′)2, a F(ab′)3, a monovalent second-specificity binding site.

18. The antibody of any one of claims 1-14, wherein the antibody is a human, humanized antibody or de-immunized antibody.

19. The antibody of any one of claims 1-14, wherein the antibody is conjugated to an imaging agent.

20. A chimeric antigen receptor comprising an antigen-binding domain at least 80% identical to an antigen-binding domain of the monoclonal antibody of any one of the preceding claims.

21. A composition comprising an antibody of any one of claims 1-14 in a pharmaceutically acceptable carrier.

22. An isolated polynucleotide molecule comprising a nucleic acid sequence encoding an antibody of any one of claims 1-14.

23. A recombinant polypeptide comprising an antibody V_{H} domain comprising CDRs 1-3 of the V_{H} domain of TREM2-Ab1Rb (SEQ ID NOs: 1, 2, and 3); CDRs 1-3 of the V_{H} domain of TREM2-Ab2Rb (SEQ ID NOs: 4, 5, and 6); CDRs 1-3 of the V_{H} domain of TREM2-Ab2Rb (SEQ ID NOs: 4, 5, and 6); CDRs 1-3 of the V_{H} domain of TREM2-Ab12Rb (SEQ ID NOs: 7, 8, and 9); CDRs 1-3 of the V_{H} domain of TREM2-Ab16Rb (SEQ ID NOs: 10, 11, and 12); CDRs 1-3 of the V_{H} domain of TREM2-Ab22Rb (SEQ ID NOs: 13, 14, and 15); CDRs 1-3 of the V_{H} domain of TREM2-Ab26Rb (SEQ ID NOs: 16, 17, and 18); CDRs 1-3 of the V_{H} domain of TREM2-Ab2Hu (SEQ ID NOs: 19, 20, and 21); CDRs 1-3 of the V_{H} domain of TREM2-Ab8Hu (SEQ ID NOs: 22, 23, and 24); or CDRs 1-3 of the V_{H} domain of TREM2-Ab119Hu (SEQ ID NOs: 25, 26, and 27).

24. A recombinant polypeptide comprising an antibody V_{L} domain comprising CDRs 1-3 of the V_{L} domain of TREM2-Ab1Rb; CDRs 1-3 of the V_{L} domain of TREM2-Ab2Rb; CDRs 1-3 of the V_{L} domain of TREM2-Ab6Rb; CDRs 1-3 of the V_{L} domain of TREM2-Ab12Rb; CDRs 1-3 of the V_{L} domain of TREM2-Ab16Rb; CDRs 1-3 of the V_{L} domain of TREM2-Ab22Rb; CDRs 1-3 of the V_{L} domain of TREM2-Ab26Rb; CDRs 1-3 of the V_{L} domain of TREM2-Ab2Hu; CDRs 1-3 of the V_{L} domain of TREM2-Ab8Hu; or CDRs 1-3 of the V_{L} domain of TREM2-Ab19Hu.

25. An isolated polynucleotide molecule comprising a nucleic acid sequence encoding a polypeptide of claim 23 or 24.

26. A host cell comprising one or more polynucleotide molecule(s) encoding an antibody of any one of claims 1-14 or a recombinant polypeptide of claim 23 or 24.

27. The host cell of claim 26, wherein the host cell is a mammalian cell, a yeast cell, a bacterial cell, a ciliate cell or an insect cell.

28. An expression vector comprising a polynucleotide having at least 95% identity to H2-Hu-HC-DNA, H8-Hu-HC-DNA, 19H-Hu HC-DNA, 16H-HC-DNA, 22H-HC-DNA, 26H-HC-DNA, H2-Hu-HC-DNA, H8-Hu-HC-DNA, or H19-Hu-HC-DNA.

29. An expression vector comprising a polynucleotide having at least 95% identity to 20L-LC-DNA, 8L-LC-DNA, 19L LC-DNA, 1K-LC-DNA. 2K-LC-DNA, 6K-LC-DNA, 2K-LC-DNA, 16K-LC-DNA, 22K-LC-DNA, or 26K-LC-DNA.

30. A method of manufacturing an antibody comprising:
(a) expressing one or more polynucleotide molecule(s) encoding a V_{L} and V_{H} chain of an antibody of any one of claims 1-14 in a cell; and
(b) purifying the antibody from the cell and/or a fluid medium in which the cell is disposed.
